(19) United States
(12) Patent Application Publication
(10) Pub. No.: US 2026/0229359 A1
(43) Pub. Date: Aug. 6, 2026

(54) METHODS AND SYSTEMS OF CLASSIFYING TUMOR TISSUE SAMPLES

(71) Applicant: Veracyte, Inc., South San Francisco, CA (US)

(72) Inventors: Yangyang Hao, San Mateo, CA (US); Jing Huang, Cupertino, CA (US); Ruochen Jiang, Irvine, CA (US); Joshua Klopper, Denver, CO (US); Yang Liu, Surrey (CA)

(21) Appl. No.: 19/420,254

(22) Filed: Dec. 15, 2025

Related U.S. Application Data
(63) Continuation of application No. PCT/US2024/032017, filed on May 31, 2024.
(60) Provisional application No. 63/508,846, filed on Jun. 16, 2023.

(51) Int. Cl. G16H 50/20

(52) U.S. Cl. CPC G16H 50/20

(57) ABSTRACT

Provided herein are methods and systems for analysing a sample of a subject by using a trained algorithm to classify the samples as invasive, non-invasive, metastatic, non-metastatic or combinations thereof. The methods may comprise using gene signatures as features of a model. The method may comprise using one or more gene aberrations as features of a model.

This application is a continuation application of International Patent Application No. PCT/US2024/032017, filed May 31, 2024, which claims the benefit of U.S. Provisional Application No. 63/508,846, filed Jun. 16, 2023, each of which is incorporated herein by reference in its entirety.

### BACKGROUND

Thyroid cancer incidence has increased substantially in the United States in recent decades, with evidence to support both an increase in detection and a true increase in occurrence. The application of cytology to thyroid nodule specimens obtained by fine-needle aspiration (FNA) biopsy may be used for diagnosis of the cancer. However, approximately one-third of thyroid nodule cytology findings today are cytologically indeterminate, and approximately three quarters of patients with cytologically indeterminate thyroid nodules have been referred for surgery, even though 80% ultimately prove to have benign nodules.

### SUMMARY

The present disclosure describes enhanced technologies for characterizing tissue samples from patients at risk of having thyroid cancer. These include improved methods for ruling out a high risk of invasiveness or metastasis in fine needle aspirate (FNA) samples from patients with an indeterminate risk of having thyroid cancer after preliminary tests.

In an aspect, the present disclosure provides a method for processing or analysing a tissue sample of a subject, comprising: (a) subjecting a first portion of said tissue sample to cytological analysis that indicates that said first portion of said tissue sample comprises a thyroid tissue sample with a risk of malignancy; (b) upon identifying said first portion of said tissue sample as comprising a thyroid tissue sample with said risk of malignancy, assaying a plurality of gene expression products from a second portion of said tissue sample to yield a first data set; (c) in a programmed computer, using a trained algorithm to process said first data set from (b) to generate a classification of said second portion of said tissue sample as having a high risk of being invasive, metastatic or a combination thereof; and (d) outputting a report indicative of said classification of said second portion of said tissue sample as having said high risk of being invasive, metastatic, or a combination thereof.

In some embodiments, the risk of malignancy is a Bethesda III, IV, V or VI classification. In some embodiments, the thyroid nodule is not benign based on cytological analysis. In some embodiments, the risk of malignancy is due to an indeterminate cytopathology. In some embodiments, the risk of malignancy is determined using a genomic sequence classifier. In some embodiments, having a high risk of being invasive comprises having an extent of invasion that is at least one of extensive lympho-vascular invasion, focal extra-thyroidal invasion, or extensive extra-thyroidal invasion. In some embodiments, having a high risk of being metastatic comprises having at least one of central neck nodes with greater than 2 mm tumor deposits, greater than 40% of lymph nodes involved, or lateral neck node metastases. In some embodiments, the assaying comprises assaying by sequencing, array hybridization, or nucleic acid amplification. In some embodiments, the classification of said second portion of said tissue sample as having a high risk of being invasive, metastatic, or a combination thereof has a negative predictive value (NPV) of at least about 90%. In some embodiments, the classification of said second portion of said tissue sample as having a high risk of being invasive, metastatic, or a combination thereof has a NPV of at least about 95%. In some embodiments, the plurality of gene expression products includes two or more of sequences corresponding to messenger ribonucleic acid (mRNA) transcripts. In some embodiments, the trained algorithm is a machine learning algorithm. In some embodiments, the machine learning algorithm is selected from the group consisting of a penalized generalized linear regression algorithm, hierarchical penalized linear regression algorithm, random forest algorithm, support vector machine algorithm, support vector machine-radial algorithm, and combinations thereof. In some embodiments, the machine learning algorithm is a random forest machine learning algorithm. In some embodiments, the machine learning algorithm is a hierarchical penalized linear regression machine learning algorithm. In some embodiments, outputting a report further comprises outputting a severity level of invasion, metastases, or combination thereof. In some embodiments, the trained algorithm processes, in said first data set, sequence information corresponding to one or more genes of Tables 3. In some embodiments, the trained algorithm processes, in said first data set, sequence information corresponding to at least genes 10 genes of Table 3. In some embodiments, the trained algorithm processes, in said first data set, sequence information corresponding to genes associated with a tumor microenvironment, drug sensitivity, or metabolism. In some embodiments, the trained algorithm processes, in said first data set, sequence information corresponding to genes associated with androgen receptors. In some embodiments, the trained algorithm processes, in said first data set, sequence information corresponding to genes associated with BRAF. In some embodiments, the trained algorithm processes, in said first data set, sequence information corresponding to genes associated with RAS. In some embodiments, the trained algorithm processes, in said first data set, sequence information corresponding to one or more genes of Table 5. In some embodiments, the trained algorithm processes, in said first data set, sequence information corresponding to one or more genes of Table 5. In some embodiments, the trained algorithm processes, in said first data set, sequence information corresponding to three or more genes of Table 5. In some embodiments, the trained algorithm processes, in said first data set, sequence information corresponding to five or more genes of Table 5. In some embodiments, the trained algorithm processes, in said first data set, sequence information corresponding to eight or more genes of Table 5. In some embodiments, the trained algorithm processes, in said first data set, sequence information corresponding to 10 or more genes of Table 5. In some embodiments, the trained algorithm processes, in said first data set, sequence information corresponding to 15 or more genes of Table 5. In some embodiments, the trained algorithm processes, in said first data set, sequence information corresponding to 17 or more genes of Table 5. In some embodiments, the trained algorithm processes, in said first data set, sequence information corresponding to 20 or more genes of Table 5. In some embodiments, the trained algorithm is trained on a plurality of gene signatures. In some embodiments, the trained algorithm processes, in said first data set, sequence information corresponding to one or more genes selected from the group consisting of CDH2, TWIST1, FN1, ITGB6, DIO1, SCL5A5, and TPO. In some embodiments, the trained algorithm processes, in said first data set, sequence information corresponding to CDH2, TWIST1, FN1, ITGB6, DIO1, SCL5A5, and TPO. In some embodiments, the plurality of gene expression products comprises gene expression products corresponding to one or genes of Table 3. In some embodiments, the plurality of gene expression products comprises gene expression products corresponding to at least 10 or more genes of Table 3. In some embodiments, the plurality of gene expression products comprises gene expression products corresponding to one or more genes of Table 5. In some embodiments, the plurality of gene expression products comprises gene expression products corresponding to at least three or more genes of Table 5. In some embodiments, the plurality of gene expression products comprises gene expression products corresponding to at least five or more genes of Table 5. In some embodiments, the plurality of gene expression products comprises gene expression products corresponding to at least eight or more genes of Table 5. In some embodiments, the plurality of gene expression products comprises gene expression products corresponding to at least ten or more genes of Table 5. In some embodiments, the plurality of gene expression products comprises gene expression products corresponding to at least 15 or more genes of Table 5. In some embodiments, the plurality of gene expression products comprises gene expression products corresponding to at least 17 or more genes of Table 5. In some embodiments, the plurality of gene expression products comprises gene expression products corresponding to at least 20 or more genes of Table 5. In some embodiments, the plurality of gene expression products comprises gene expression products corresponding to one or more genes selected from the group consisting of CDH2, TWIST1, FN1, ITGB6, DIO1, SCL5A5, and TPO. In some embodiments, the tissue sample is a thyroid tissue sample. In some embodiments, the tissue sample is a fresh frozen sample. In some embodiments, the tissue sample is a needle aspirate sample. In some embodiments, the needle aspirate sample is a fine needle aspirate sample. In some embodiments, the trained algorithm has been trained with a training set of samples, and wherein said tissue sample is independent of said training set of samples.

In an aspect, the present disclosure provides a method for processing or analyzing a tissue sample of a subject, comprising: (a) subjecting a first portion of said tissue sample to cytological analysis that indicates that said first portion of said tissue sample has a risk of malignancy; (b) upon identifying said first portion of said tissue sample as having a risk of malignancy, assaying a plurality of gene expression products from a second portion of said tissue sample to yield a first data set; (c) in a programmed computer, using a machine learning algorithm that processes said first data set from (b) to generate a classification of said second portion of said tissue sample as having a high risk of being invasive, metastatic or a combination thereof; and (d) outputting a report indicative of said classification of said second portion of said tissue sample as having a high risk of being invasive, metastatic, or a combination thereof. In some embodiments, the machine learning algorithm comprises a penalized generalized linear regression algorithm, hierarchical penalized linear regression algorithm, random forest algorithm, support vector machine algorithm, support vector machine-radial algorithm, and combinations thereof. In some embodiments, the machine learning algorithm is a random forest machine learning algorithm. In some embodiments, the machine learning algorithm is a hierarchical penalized linear regression machine learning algorithm. In some embodiments, the risk of malignancy is a Bethesda III, IV, V or VI classification. In some embodiments, the thyroid nodule is not benign based on cytological analysis. In some embodiments, the risk of malignancy is due to an indeterminate cytopathology. In some embodiments, the risk of malignancy is determined using a genomic sequence classifier. In some embodiments, the having a high risk of being invasive comprises having an extent of invasion that is at least one of extensive lympho-vascular invasion, focal extra-thyroidal invasion, or extensive extra-thyroidal invasion. In some embodiments, the assaying comprises assaying by sequencing, array hybridization, or nucleic acid amplification. In some embodiments, the classification of said second portion of said tissue sample as having a high risk of being invasive, metastatic, or a combination thereof has a NPV of at least about 90%. In some embodiments, the classification of said second portion of said tissue sample as having a high risk of being invasive, metastatic, or a combination thereof has a NPV of at least about 95%. In some embodiments, the plurality of gene expression products includes two or more of sequences corresponding to mRNA transcripts. In some embodiments, the trained algorithm is a machine learning algorithm. In some embodiments, outputting a report further comprises outputting a severity level of invasion. In some embodiments, the trained algorithm processes, in said first data set, sequence information corresponding to genes associated with a tumor microenvironment, drug sensitivity, or metabolism. In some embodiments, the trained algorithm processes, in said first data set, sequence information corresponding to genes associated with androgen receptors. In some embodiments, the trained algorithm processes, in said first data set, sequence information corresponding to genes associated with BRAF. In some embodiments, the trained algorithm processes, in said first data set, sequence information corresponding to genes associated with RAS. In some embodiments, the trained algorithm processes, in said first data set, sequence information corresponding to one or more genes of Table 5. In some embodiments, the trained algorithm processes, in said first data set, sequence information corresponding to one or more genes of Table 5. In some embodiments, the trained algorithm processes, in said first data set, sequence information corresponding to three or more genes of Table 5. In some embodiments, the trained algorithm processes, in said first data set, sequence information corresponding to five or more genes of Table 5. In some embodiments, the trained algorithm processes, in said first data set, sequence information corresponding to eight or more genes of Table 5. In some embodiments, the trained algorithm processes, in said first data set, sequence information corresponding to 10 or more genes of Table 5. In some embodiments, the trained algorithm processes, in said first data set, sequence information corresponding to 15 or more genes of Table 5. In some embodiments, the trained algorithm processes, in said first data set, sequence information corresponding to 17 or more genes of Table 5. In some embodiments, the trained algorithm processes, in said first data set, sequence information corresponding to 20 or more genes of Table 5. In some embodiments, the trained algorithm is trained on a plurality of gene signatures. In some embodiments, the trained algorithm processes, in said first data set, sequence information corresponding to one or more genes selected from the group consisting of CDH2, TWIST1, FN1, ITGB6, DIO1, SCL5A5, and TPO. In some embodiments, the trained algorithm processes, in said first data set, sequence information corresponding to CDH2, TWIST1, FN1, ITGB6, DIO1, SCL5A5, and TPO. In some embodiments, the plurality of gene expression products comprises gene expression products corresponding to one or genes of Table 3. In some embodiments, the plurality of gene expression products comprises gene expression products corresponding to at least 10 or more genes of Table 3. In some embodiments, the plurality of gene expression products comprises gene expression products corresponding to one or more genes of Table 5. In some embodiments, the plurality of gene expression products comprises gene expression products corresponding to at least three or more genes of Table 5. In some embodiments, the plurality of gene expression products comprises gene expression products corresponding to at least five or more genes of Table 5. In some embodiments, the plurality of gene expression products comprises gene expression products corresponding to at least eight or more genes of Table 5. In some embodiments, the plurality of gene expression products comprises gene expression products corresponding to at least ten or more genes of Table 5. In some embodiments, the plurality of gene expression products comprises gene expression products corresponding to at least 15 or more genes of Table 5. In some embodiments, the plurality of gene expression products comprises gene expression products corresponding to at least 17 or more genes of Table 5. In some embodiments, the plurality of gene expression products comprises gene expression products corresponding to at least 20 or more genes of Table 5. In some embodiments, the plurality of gene expression products comprises gene expression products corresponding to one or more genes selected from the group consisting of CDH2, TWIST1, FN1, ITGB6, DIO1, SCL5A5, and TPO. In some embodiments, the trained algorithm is trained on a plurality of gene signatures. In some embodiments, the tissue sample is a thyroid tissue sample. In some embodiments, the tissue sample is a fresh frozen sample. In some embodiments, the tissue sample is a needle aspirate sample. In some embodiments, the needle aspirate sample is a fine needle aspirate sample. In some embodiments, the trained algorithm has been trained with a training set of samples, and wherein said tissue sample is independent of said training set of samples.

In an aspect, the present disclosure provides a method for diagnosing the risk of invasive and/or metastatic thyroid cancer in a subject comprising the steps of: (i) assaying a plurality of gene expression products from a thyroid tissue sample of the subject, said sample being identified as at risk of malignancy; and (ii) classifying the risk of the subject to have invasive and/or metastatic thyroid cancer based on the results of step (i). In some embodiments, the method comprises the step of identifying the risk of malignancy of said sample, preferably by subjecting the thyroid tissue sample to cytological analysis; this identification step can be performed prior to or concomitantly to step (i). In some embodiments, classifying the risk of the subject to have invasive and/or metastatic thyroid cancer is based on the results of step (i) and the risk of malignancy. In some embodiments, the risk of malignancy is a Bethesda III, IV, V or VI classification. In some embodiments, the thyroid nodule is not benign based on cytological analysis. In some embodiments, the risk of malignancy is due to an indeterminate cytopathology. In some embodiments, the risk of malignancy is determined using a genomic sequence classifier. In some embodiments, the having a high risk of being invasive comprises having an extent of invasion that is at least one of extensive lympho-vascular invasion, focal extra-thyroidal invasion, or extensive extra-thyroidal invasion. In some embodiments, the assaying comprises sequencing, array hybridization, or measuring expression level (such as by nucleic acid amplification). In some embodiments, the gene expression products are mRNA transcripts such at least one, preferably at least 10, mRNA transcripts corresponding to the genes of Table 3. In some embodiments, step (i) of assaying a plurality of gene expression products provides sequence information, such as mRNA or gene expression, mitochondrial transcripts, genetic variants and/or fusion transcripts; or, in other words, the results of step (i) comprise sequence information, such as mRNA or gene expression, mitochondrial transcripts, genetic variants and/or fusion transcripts. In some embodiments, sequence information is gene expression level and/or sequence variants (i.e., genetic variants). In some embodiments, the sequence information corresponds to one or more genes of Tables 3. In some embodiments, the sequence information corresponds to at least genes 10 genes of Table 3. In some embodiments, the sequence information corresponds to genes associated with a tumor microenvironment, drug sensitivity, or metabolism. In some embodiments, the sequence information corresponds to genes associated with androgen receptors. In some embodiments, the sequence information corresponds to genes associated with BRAF. In some embodiments, the sequence information corresponds to genes associated with RAS. In some embodiments, the thyroid tissue sample is a fresh frozen sample. In some embodiments, the thyroid tissue sample is a needle aspirate sample. In some embodiments, the needle aspirate sample is a fine needle aspirate sample. In some embodiments, the classification of said tissue sample as having a high risk of being invasive, metastatic, or a combination thereof has a NPV of at least about 90%. In some embodiments, the classification of said tissue sample as having a high risk of being invasive, metastatic, or a combination thereof has a NPV of at least about 95%. In some embodiments, the classifying step (ii) comprises processing or analysing the results of step (i) with a trained algorithm on a computer. In some embodiments, the classifying step (ii) comprises processing or analysing the results of step (i) and the risk of malignancy with a trained algorithm on a computer. In some embodiments, the trained algorithm is a machine learning algorithm which may be a penalized generalized linear regression algorithm, a hierarchical penalized linear regression algorithm, a random forest algorithm, a support vector machine algorithm, a support vector machine-radial algorithm, or any combination thereof. In some embodiments, the machine learning algorithm is a random forest machine learning algorithm. In some embodiments, the machine learning algorithm is a hierarchical penalized linear regression machine learning algorithm. In some embodiments, the trained algorithm processes sequence information corresponding to one or more genes of Tables 3. In some embodiments, the trained algorithm analyses sequence information corresponding to at least genes 10 genes of Table 3. In some embodiments, the trained algorithm processes sequence information corresponding to genes associated with a tumor microenvironment, drug sensitivity, or metabolism. In some embodiments, the trained algorithm processes sequence information corresponding to genes associated with androgen receptors. In some embodiments, the trained algorithm processes sequence information corresponding to genes associated with BRAF. In some embodiments, the trained algorithm processes sequence information corresponding to genes associated with RAS. In some embodiments, the trained algorithm is trained on a plurality of gene signatures. In some embodiments, the trained algorithm has been trained with a training set of thyroid tissue samples, and wherein said tissue sample is independent of said training set of samples. In some embodiments, the method further comprises, after step (ii), the step of outputting a report indicative of the risk of the subject to have invasive and/or metastatic thyroid cancer. In some embodiments, the reports are further indicative of the severity level of invasion.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an overview of the signatures that were used to rule out high risk of invasion or metastasis.

FIG. 2 shows an overview of the parameters used to develop greater than 400 machine learning models after repeated nested cross-validation in order to find those models with reliable performance estimations.

FIG. 3 shows the rules used to label fine needle aspirate (FNA) biopsy samples for extent of invasion and extent of metastases.

FIG. 4 shows an overview of the method used to build mRNA expression-based risk signatures with a high negative predictive value for ruling out invasion or metastasis.

FIG. 5 shows an example of how different models for ruling out invasiveness were evaluated relative to each other based on the rule out percentage when a particular set of features selection algorithms and machine learning models was used.

FIG. 6 shows an example of how different models for ruling out metastasis were evaluated relative to each other based on the rule out percentage when a particular set of features selection algorithms and machine learning models was used.

FIGS. 7A-7B shows best performing signature models for invasion (FIG. 7A) and metastasis (FIG. 7B).

FIG. 8 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about 1, 2, 5, 10, 20, 30, 40, 50, 60, 65, 70, 75, or about 80 years of age. The subject may have a disease (e.g., thyroid cancer). The subject may be asymptomatic with respect to a disease. The subject may have or be suspected of having a disease, such as cancer. The subject may be a patient, such as a patient being treated for a disease, such as a cancer patient. The subject may be predisposed to a risk of developing a disease such as cancer. The subject may be in remission from a disease, such as a cancer patient. The subject may be healthy.

The term “disease,” as used herein, generally refers to any abnormal or pathologic condition that affects a subject. Examples of a disease include cancer, such as, for example, thyroid cancer, parathyroid cancer, lung cancer, skin cancer, and others. The disease may be a disease comprising conditions of abnormal growth in one or more tissues of a subject including but not limited to skin, heart, thyroid, lung, kidney, breast, pancreas, liver, muscle, smooth muscle, bladder, gall bladder, colon, intestine, brain, esophagus, or prostate. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein.

The term “sequence variant,” “sequence variation,” “sequence alteration” or “allelic variant,” as used herein, generally refer to a specific change or variation in relation to a reference sequence, such as a genomic deoxyribonucleic acid (DNA) reference sequence, a coding DNA reference sequence, or a protein reference sequence, or others. The reference DNA sequence can be obtained from a reference database. A sequence variant may affect function. A sequence variant may not affect function. A sequence variant can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a sequence variation can include a substitution, a deletion, an insertion, an inversion, or a conversion in one or more nucleotides or one or more amino acids. A sequence variant may be a point mutation. A sequence variant may be a fusion gene. A fusion pair or a fusion gene may result from a sequence variant, such as a translocation, an interstitial deletion, a chromosomal inversion, or any combination thereof. A sequence variation can constitute variability in the number of repeated sequences, such as triplications, quadruplications, or others. For example, a sequence variation can be an increase or a decrease in a copy number associated with a given sequence (i.e., copy number variation, or CNV). A sequence variation can include two or more sequence changes in different alleles or two or more sequence changes in one allele. A sequence variation can include two different nucleotides at one position in one allele, such as a mosaic. A sequence variation can include two different nucleotides at one position in one allele, such as a chimeric. A sequence variant may be present in a malignant tissue. A sequence variant may be present in a benign tissue. Absence of a variant may indicate that a tissue or sample is benign. As an alternative, absence of a variant may not indicate that a tissue or sample is benign.

The term “disease diagnostic,” as used herein, generally refers to identifying, diagnosing or screening for a disease, to stratify a risk of occurrence of a disease, to monitor progression or remission of a disease, to formulate a treatment regime for the disease, or any combination thereof. A disease diagnostic can include a) obtaining information from one or more tissue samples from a subject, b) making a determination about whether the subject has a particular disease based on the information or tissue sample obtained, c) stratifying the risk of occurrence of the disease in the subject, d) confirming whether a subject has the disease, is developing the disease, or is in disease remission, or any combination thereof. The disease diagnostic may inform a particular treatment or therapeutic intervention for the disease. The disease diagnostic may also provide a score indicating for example, the severity or grade of a disease such as cancer, or the likelihood of an accurate diagnosis, such as via a p-value, a corrected p-value, or a statistical confidence indicator. The disease diagnostic may also indicate a particular type of a disease. For example, a disease diagnostic for thyroid cancer may indicate a subtype such as follicular adenoma (FA), nodular hyperplasia (NHP), lymphocytic thyroiditis (LCT), Hürthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), anaplastic thyroid carcinoma (ATC), renal carcinoma (RCC), breast carcinoma (BCA), melanoma (MMN), B cell lymphoma (BCL), parathyroid (PTA), or hyperplasia papillary carcinoma (HPC).

The term “high risk,” as used herein, generally refers to a relatively high likelihood of developing or exhibiting a condition (e.g., invasive growth or metastasis). For example, high risk of invasiveness may be a high likelihood of developing or exhibiting invasive growth by samples due to the cells in the samples already exhibiting extensive extra-thyroidal or lympho-vascular or focal extra-thyroidal growth or being classified with samples that have cells with such growth after evaluation of gene expression by a trained machine learning algorithm.

“Low risk” generally refers to a relatively low likelihood of developing or exhibiting a condition (e.g., invasive growth or metastasis). For example, low risk of invasiveness may be a low likelihood of developing or exhibiting invasive growth due to the cells already exhibiting no signs of invasion or else minor lympho-vascular or transcapular growth or being classified with samples that have cells with such growth after evaluation by a trained machine learning algorithm. Intermediate risk may refer to a likelihood of developing or exhibiting a condition that is between high risk and low risk.

Thyroid cancer incidence has increased substantially in the United States in recent decades, with evidence to support both an increase in detection and a true increase in occurrence. The application of cytology to thyroid nodule specimens obtained by fine-needle aspiration (FNA) biopsy can help with patient management by reducing surgery by one half and doubling the proportion of cancer among patients who ultimately underwent surgery. However, it has been observed that approximately one-third of thyroid nodule cytology findings today are cytologically indeterminate, and approximately three quarters of patients with cytologically indeterminate thyroid nodules have been referred for surgery, even though 80% ultimately prove to have benign nodules.

Several tests have been developed to identify variants and fusions within patient FNA biopsy samples in order to better predict whether a cytologically indeterminate thyroid nodule is benign or malignant. Nonetheless, improvements in risk stratification are still needed. For example, the 2015 American Thyroid Association thyroid cancer risk stratification system is driven primarily by the extent of vascular and extrathyroidal extension, as well as metastases. Furthermore, focus group findings suggest that physicians find that reporting risk of metastasis is clinically relevant because physicians are more likely to order a test that reports such a risk.

While expression signatures of specific molecular events and pathways have been discovered to be associated with invasion or metastases, composite expression signatures taking into consideration various molecular events and pathways that better stratify and rule out high risk of invasion or metastases are still lacking. In addition, these previously used expression signatures are derived and evaluated on formalin fixed paraffin embedded (FFPE) tissue samples and their generalizability to fresh frozen FNA biopsies—a specimen type widely collected for thyroid tumors—are unknown.

### Methods for Generating Classification for Tissue Samples for a Disease

The present disclosure provides methods and systems for processing or analysing a tissue sample of a subject to generate a classification of tissue sample. These methods and systems may classify samples as invasive, non-invasive, or metastatic or non-metastatic. As such the methods and system herein may allow for improved diagnostic and treatment of cancer and associated tumors. For example, identification of a tissue as metastatic or invasive may allow for a physician to perform or recommend a treatment regimen suitable for metastatic or invasive tissue. For example, a surgical removal of the tissue or treatment with a chemotherapeutic agent may be performed Similarly, upon identification of a tissue as non-metastatic or non-invasive may allow for a physician to perform or recommend a treatment regimen suitable for non-metastatic tissue. For example, a patient with a non-metastatic or non-invasive tissue may be spared from surgery or recommended to undergo a less invasive procedure or less aggressive treatment regimen.

Methods described in the present disclosure may have improved specificity for identification of benign nodules and maintained high sensitivity for determination of tumors as invasive or metastatic, which may spare even more patients from surgery with an accurate benign genomic result (negative predictive value [NPV]) and increase the cancer yield among those with a suspicious result (positive predictive value [PPV]).

The methods may comprise obtaining a plurality of gene expression products from a cytologically indeterminate tissue sample and using an algorithm to analyze the gene expression products to classify the tissue samples as invasive or non-invasive, or metastatic or non-metastatic. Some techniques for using preoperative genomic information for thyroid nodule differential diagnosis may involve use messenger RNA (“mRNA”) transcript expression levels to categorize cytologically indeterminate samples. Altered messenger RNA expression can occur for several reasons, including complex upstream interactions that occur because of sequence changes in key core genes or in relevant peripheral genes, the effect of epigenetic changes that occur without DNA sequence alterations, and both internal and external modifiers, such as inflammation and lifestyle or environment.

The present disclosure describes enhanced technologies for characterizing genomic information, including improved methods for the measurement of RNA transcriptome expression and sequencing of nuclear and mitochondrial RNAs, measurement changes in genomic copy number, including loss of heterozygosity, and the development of enhanced bioinformatics and machine learning strategies, resulting in a more robust genomic test.

The methods provided herein may comprises assaying gene expression products. A plurality of gene expression products comprises sequences may correspond to mRNA transcripts, mitochondrial transcripts, chromosomal loss of heterozygosity, DNA variants and/or fusion transcripts. The methods may comprise assaying gene expression products corresponding to one or more genes. For example, the methods may comprise assaying gene expression products corresponding to one or more genes of Table 3. For example, the methods may comprise assaying gene expression products corresponding to at least 5 genes of Table 3. For example, the methods may comprise assaying gene expression products corresponding to at least 5 genes of Table 3. For example, the methods may comprise assaying gene expression products corresponding to at least 10 genes of Table 3. For example, the methods may comprise assaying gene expression products corresponding to at least 20 genes of Table 3. The methods may comprise assaying gene expression products corresponding to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of Table 3. For example, the methods may comprise assaying gene expression products corresponding to one or more genes of Table 5. For example, the methods may comprise assaying gene expression products corresponding to at least 5 genes of Table 5. For example, the methods may comprise assaying gene expression products corresponding to at least 10 genes of Table 5. For example, the methods may comprise assaying gene expression products corresponding to at least 20 genes of Table 5. The methods may comprise assaying gene expression products corresponding to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more genes of Table 5. The methods may comprise assaying gene expression products corresponding to at least one or more genes selected from the group consisting of CDH2, TWIST1, FN1, ITGB6, DIO1, SCL5A5, and TPO. The methods may comprise assaying gene expression products corresponding to at least two or more genes selected from the group consisting of CDH2, TWIST1, FN1, ITGB6, DIO1, SCL5A5, and TPO. The methods may comprise assaying gene expression products corresponding to at least three or more genes selected from the group consisting of CDH2, TWIST1, FN1, ITGB6, DIO1, SCL5A5, and TPO. The methods may comprise assaying gene expression products corresponding to at least four or more genes selected from the group consisting of CDH2, TWIST1, FN1, ITGB6, DIO1, SCL5A5, and TPO. The methods may comprise assaying gene expression products corresponding to at least five or more genes selected from the group consisting of CDH2, TWIST1, FN1, ITGB6, DIO1, SCL5A5, and TPO. The methods may comprise assaying gene expression products corresponding to at least six or more genes selected from the group consisting of CDH2, TWIST1, FN1, ITGB6, DIO1, SCL5A5, and TPO. The methods may comprise assaying gene expression products corresponding to CDH2, TWIST1, FN1, ITGB6, DIO1, SCL5A5, and TPO.

In some examples, the method uses a trained algorithm that is implemented by one or more programmed computer processors to analyze the expression gene products to generate a classification of tissue sample as invasive or non-invasive. In some examples, the method uses a trained algorithm that is implemented by one or more programmed computer processors to analyze the expression gene products to generate a classification of tissue sample as metastatic or non-metastatic. The classification of a tissue sample may be a classification relating to the severity of a metric. For example, the classification may be that a tissue sample is highly metastatic as compared to slightly metastatic. The algorithm may be a trained algorithm (e.g., an algorithm that is trained on at least 10, 200, 100 or 500 reference samples). References samples may be obtained from subjects having been diagnosed with the disease or from healthy subjects. The trained algorithm may process the sequence information of expression gene products. For example, the trained algorithm may process sequence information or expression levels corresponding to one of more genes. The trained algorithm may process sequence information or expression levels corresponding at least 1, 2, 3, 4, 5, 6, 7, 8, 9, 10, 11, 12, 13, 14, 15, 16, 17, 19, 20, 21, 22, 23, 24, 25, 26, 27, 28, 29, 30, 40, 50, 60, 70, 80, 90, 100, 120, 140, 160, 180, 200, 220, 240, 260, 280, 300, 400, 500, 600,700, 800, 900, 1000, 2000, 3000, 4000, 5000, 6000, 7000, 8000, 9000, 10000, or more genes. The trained algorithm may analyze the sequence information of expression gene products or expression levels of genes provided in Table 3. The trained algorithm may process sequence information or expression levels corresponding to one or more of genes of Table 5. The trained algorithm may process sequence information or expression levels corresponding to one or more of genes selected from the group consisting of CDH2, TWIST1, FN1, ITGB6, DIO1, SCL5A5, and TPO.

As set forth in the present disclosure, an expression level of one or more genes of gene expression products can be obtained by assaying for an expression level. Assaying may comprise array hybridization, nucleic acid sequencing, nucleic acid amplification, or others. Assaying may comprise sequencing, such as DNA or RNA sequencing. Such sequencing may be by next generation (NextGen) sequencing, such as high throughput sequencing or whole genome sequencing (e.g., Illumina). Such sequencing may include enrichment. Assaying may comprise reverse transcription polymerase chain reaction (PCR). Assaying may utilize markers, such as primers, which are selected for each of the one or more genes.

Additional methods for determining gene expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immunohistochemistry, blotting, sequencing, RNA sequencing, DNA sequencing (e.g., sequencing of complementary deoxyribonucleic acid (cDNA) obtained from RNA); next generation (Next-Gen) sequencing (e.g., Illumina, Pacific Biosciences of California), nanopore sequencing, pyrosequencing, or Nanostring sequencing. Gene expression product levels may be normalized to an internal standard such as total messenger ribonucleic acid (mRNA) or the expression level of a particular gene.

The methods disclosed herein may include extracting and analyzing protein or nucleic acid (RNA or DNA) from one or more samples from a subject. Nucleic acids can be extracted from the entire sample obtained or can be extracted from a portion. In some cases, the portion of the sample not subjected to nucleic acid extraction may be analyzed by cytological examination or immunohistochemistry. Methods for RNA or DNA extraction from biological samples can include for example phenol-chloroform extraction (such as guanidinium thiocyanate phenol-chloroform extraction), ethanol precipitation, spin column-based purification, or others.

The sample obtained from the subject may be cytologically ambiguous or suspicious (or indeterminate). In some cases, the sample may be suggestive of the presence of a disease. The volume of sample obtained from the subject may be small, such as about 100 microliters, 50 microliters, 10 microliters, 5 microliters, 1 microliter or less. The sample may comprise a low quantity or quality of polynucleotides, such as a tissue sample with degraded or partially degraded RNA. For example, an FNA sample may yield low quantity or quality of polynucleotides. In such examples, the RNA Integrity Number (RIN) value of the sample may be about 9.0 or less. In some examples, the RIN value may be about 6.0 or less.

In various aspects, the samples obtained from a subject may have a risk of malignancy. The sample may be diagnosed as malignant; however, it may be difficult to ascertain if the tumor will be invasive or metastasize. The risk of malignancy may be a Bethesda III, IV, V or VI classification. The Bethesda System for Reporting Thyroid Cytopathology (TBSRTC) established a standardized reporting system with a limited number of diagnostic categories for thyroid fine-needle aspiration (FNA) specimens. For example, a Bethesda category III corresponds to “Atypia of Undetermined Significance or Follicular Lesion of Undetermined Significance,” a Bethesda category IV corresponds to a “Follicular Neoplasm or Suspicious for a Follicular Neoplasm”, a Bethesda category V corresponds to “Suspicious for Malignancy,” and a Bethesda category VI corresponds to “Malignant”. The risk of malignancy may be due to an indeterminate cytopathology. The risk of malignancy may be determined using a classifier. The risk of malignancy may be determined using a classifier. For example, the classifier may be trained algorithm classifier. The classifier may be a genomic sequence classifier. The classifier may analyze one or more gene or variant expression products to generate a risk of malignancy. For example, the risk of malignancy may be determined using the Afirma genomic sequence classifier. See, e.g., Krane et al. (2020) “The Afirma Xpression Atlas for thyroid nodules and thyroid cancer metastases: Insights to inform clinical decision-making from a fine-needle aspiration sample” Cancer Cytopathol 128 (7): 452-9, which is entirely incorporated herein by reference.

The use of the methods described herein may be used in addition to other studies to analyze the sample. For example, the sample may be previously analyzed using cytological, or histological methods. For example, the sample may be previously analyze using methods observing nucleic acids derived from cell. For example, the sample may be previously analyzed for gene expression, gene expression levels, the presence of genetic aberrations.

### Risk of Invasion or Metastasis

In some cases, the methods disclosed herein further comprise processing the gene expression products using a curated panel of sequence associated with variants and/or fusions and which includes well validated variants and variants whose clinical significance is emerging to provide further genomic information on samples identified as being invasive or metastatic. In some examples, this may include identifying one or more genes, genetic aberrations of the one or more genes, or other genomic information disclosed in, for example, U.S. Pat. No. 8,541,170, U.S. Patent Publication No. 2018/0016642, and U.S. Patent Publication No. 2020/0232046, each of which is entirely incorporated herein by reference.

The genetic aberrations may be validated or may have emerging clinical significance. The risk of invasiveness may characterize one or more genetic aberrations as (1) highly associated with invasive nodules, (2) associated with both non-invasive and invasive nodules, or (3) as having insufficient published evidence to characterize such risk. The risk of metastases may characterize one or more genetic aberrations as (1) highly associated with metastatic nodules, (2) associated with both non-metastatic and metastatic nodules, or (3) as having insufficient published evidence to characterize such risk.

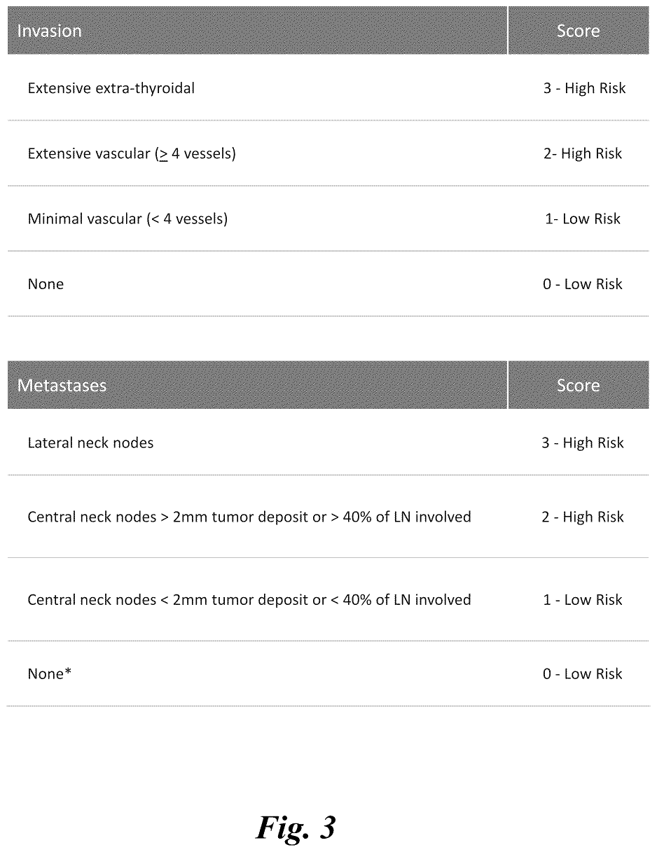

The methods disclosed herein comprise identifying one or more genetic aberrations in a sample to indicate the level of risk of invasion or metastases associated with the genetic aberration. Prognostic information may comprise TNM stage and American Thyroid Association (ATA) risk. ATA risk of invasion or metastases comprises risk categories 1-3 which may correspond to low risk or high risk. FIG. 3 shows a categorization of risk and associated cytology. Identification of one or more genetic aberrations may increase the risk of invasion or metastasis reported by one or more classifiers as used in the methods disclosed herein. Identification of one or more genetic aberrations may not increase the risk of invasion or metastasis reported by one or more classifiers as used in the methods disclosed herein. A reported risk of invasion or metastasis generated by one or more classifiers of the present disclosure may not be reduced in some cases where no genetic aberrations in one or more genes of Table 3 are identified.

Various aspects provided in this disclosure comprise generation of data and analysis of the genetic data (e.g., expression level of genes or the presence of a sequence variant). The methods may comprise using data corresponding to one or more genes. For example, the methods may comprise analysis of data corresponding to one or more genes of Table 3. For example, the methods may comprise analysis of data corresponding to at least 5 genes of Table 3. For example, the methods may comprise analysis of data corresponding to at least 5 genes of Table 3. For example, the methods may comprise analysis of data corresponding to at least 10 genes of Table 3. For example, the methods may comprise analysis of data corresponding to at least 20 genes of Table 3. The methods may comprise analysis of data corresponding to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more of Table 3. For example, the methods may comprise analysis of data corresponding to one or more genes of Table 5. For example, the methods may comprise analysis of data corresponding to at least 5 genes of Table 5. For example, the methods may comprise analysis of data corresponding to at least 10 genes of Table 5. For example, the methods may comprise analysis of data corresponding to at least 20 genes of Table 5. The methods may comprises analysis of data corresponding to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more genes of Table 5. The methods may comprise analysis of data corresponding to at least one or more genes selected from the group consisting of CDH2, TWIST1, FN1, ITGB6, DIO1, SCL5A5, and TPO. The methods may comprise analysis of data corresponding to at least two or more genes selected from the group consisting of CDH2, TWIST1, FN1, ITGB6, DIO1, SCL5A5, and TPO. The methods may comprise analysis of data corresponding to at least three or more genes selected from the group consisting of CDH2, TWIST1, FN1, ITGB6, DIO1, SCL5A5, and TPO. The methods may comprise analysis of data corresponding to at least four or more genes selected from the group consisting of CDH2, TWIST1, FN1, ITGB6, DIO1, SCL5A5, and TPO. The methods may comprise analysis of data corresponding to at least five or more genes selected from the group consisting of CDH2, TWIST1, FN1, ITGB6, DIO1, SCL5A5, and TPO. The methods may comprise analysis of data corresponding to at least six or more genes selected from the group consisting of CDH2, TWIST1, FN1, ITGB6, DIO1, SCL5A5, and TPO. The methods may comprise analysis of data corresponding to CDH2, TWIST1, FN1, ITGB6, DIO1, SCL5A5, and TPO. The data corresponding to one or more genes may be used (e.g., as feature) or processed with a trained algorithm, for example, to classify a tissue.

The methods may comprise analysis or generation of data corresponding to one or more gene signatures. A gene signature may comprise a set of genes that are associated with an attribute (such as a phenotype), a metabolic or cell signalling pathway, or disease state, or a set of otherwise related genes. The gene signatures may comprise a measure of the expression of 1 to 20,000 genes. In some instances, a signature comprises a measure of the expression of 1, 2, 3, 4, or 5 genes. In some instances, a signature comprises a measure of the expression of 6, 7, 8, 9, or 10 genes. In some instances, a signature comprises a measure of the expression of 10 to 100 genes. In some instances, a signature comprises a measure of the expression of more than 100 to 200 genes, 200 to 300 genes, 300 to 400 genes, 400 to 500 genes, 500 to 600 genes, 600 to 700 genes, 700 to 800 genes, 800 to 900 genes, or 900 to 1000 genes. In some instances, a signature comprises a measure of the expression of 1000 to 2000 genes. In some instances, a signature comprises a measure of the expression of more than 2000 genes. In some instances, a signature comprises a measure of the expression of more than 3000 genes. In some instances, a signature comprises a measure of the expression of more than 4000 genes. In some instances, a signature comprises a measure of the expression of more than 5000 genes. In some instances, a signature comprises a measure of the expression of 5000 to 10,000 genes. In some instances, a signature comprises a measure of the expression of 10,000 to 11,000 genes, 11,000 to 12,000 genes, 12,000 to 13,000 genes, 13,000 to 14,000 genes, 14,000 to 15,000 genes, 15,000 to 16,000 genes, 16,000 to 17,000 genes, 17,000 to 18,000 genes, 18,000 to 19,000 genes, or 19,000 to 20,000 genes. As described elsewhere in this disclosure, the gene signatures may be used (e.g., as feature) or processed with a trained algorithm to classify a tissue.

Data corresponding to one or more genes may be expression level data. Data corresponding to one or more genes may be processed to generate a score. The score may be a composite score. The composite score may combine data from multiple genes to generate a score representative of the multiple genes. For example, the average expression level or two or more genes may be determined to generate a composite score. The score (e.g., composite score) may be processed using a trained algorithm.

### Risk of Malignancy Using Afirma

In various embodiments, the tissue may initially be classified as having a risk of malignancy. In some cases, the methods disclosed herein further comprise processing the gene expression products using an a curated panel of sequence associated with variants and/or fusions and which includes well validated variants and variants whose clinical significance is emerging (such as, for example the Afirma assay to provide further genomic information on samples identified as being suspicious for malignancy, or malignant, the method comprising identifying any one of the genetic aberrations in the sample to indicate (i) risk of malignancy, (ii) a histological subtype, and (iii) prognosis associated with each of the genetic aberration identified in the sample. In some examples, this may include identifying one or more genes, genetic aberrations of the one or more genes, or other genomic information disclosed in, for example, U.S. Pat. No. 8,541,170, U.S. Patent Publication No. 2018/0016642, and U.S. Patent Publication No. 2020/0232046, each of which is entirely incorporated herein by reference.

The genetic aberrations may be validated or may have emerging clinical significance. The risk of malignancy may characterize one or more genetic aberrations as (1) highly associated with malignant nodules, (2) associated with both benign and malignant nodules, or (3) as having insufficient published evidence to characterize such risk.

The methods disclosed herein provide identifying one or more genetic aberrations in a sample that are indicative of a histological subtype. Histological subtypes may include classical parathyroid cancer (cPTC), infiltrative follicular variant of papillary thyroid carcinoma (infiltrative FVPTC), noninvasive encapsulated FVPTC (EFVPTC), Follicular thyroid carcinoma (FTC), and/or follicular adenomas (FA).

The methods disclosed herein comprise identifying one or more genetic aberrations in a sample to indicate prognosis associated with the genetic aberration. Prognostic information may comprise TNM stage and American Thyroid Association (ATA) risk. The TNM Staging System is based on the extent of the tumor (T), the extent of spread to the lymph nodes (N), and the presence of metastasis (M). The T category describes the original (primary) tumor. The TNM stage may comprise stages 1-4. ATA risk of recurrence staging system may comprises risk categories 1-3 which may correspond to low, intermediate, or high risk. The 761 nucleotide variant panel may have a PPA rate of at least 70%, 75%, 80%, 85%, 86%, 87%, 88%, 89%, 90%, 91%, 92%, 93%, 94%, 95%, 96%, 97%, 98%, 99%, or more. The 130 fusion panel may have a PPA rate of at least 70%, 75%, 80%, 85%, 86%, 87%, 88%, 89%, 90%, 91%, 92%, 93%, 94%, 95%, 96%, 97%, 98%, 99%, or more. Identification of one or more genetic aberrations may increase the risk of malignancy reported by one or more classifiers as used in the methods disclosed herein. Identification of one or more genetic aberrations may not increase the risk of malignancy reported by one or more classifiers as used in the methods disclosed herein. A reported risk of malignancy generated by one or more classifiers of the present disclosure may not be reduced in some cases where no genetic aberrations in one or more genes are identified.

### Samples

A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, serum, plasma, urine, cerebrospinal fluid, seminal fluid, saliva, sputum, stool, lymph fluid, tissue, or any combination thereof. A sample can be a tissue-specific sample such as a sample obtained from a thyroid, skin, heart, lung, kidney, breast, pancreas, liver, muscle, smooth muscle, bladder, gall bladder, colon, intestine, brain, esophagus, or prostate.

The sample may be obtained by various approaches, such as the biopsy methods provided herein, swabbing, scraping, phlebotomy, or other approaches. A sample of the present disclosure can be obtained by various methods, such as, for example, fine needle aspiration (FNA), core needle biopsy, vacuum assisted biopsy, incisional biopsy, excisional biopsy, punch biopsy, shave biopsy, skin biopsy, or any combination thereof. The sample may be obtained by non-invasive methods including but not limited to: scraping of the skin or cervix, swabbing of the cheek, saliva collection, urine collection, feces collection, collection of menses, tears, or semen.

FNA, also referred to as fine needle aspirate biopsy (FNAB), or needle aspirate biopsy (NAB), is a method of obtaining a small amount of tissue from a subject. FNA can be less invasive than a tissue biopsy, which may require surgery and hospitalization of the subject to obtain the tissue biopsy. The needle of a FNA method can be inserted into a tissue mass of a subject to obtain an amount of sample for further analysis. In some cases, two needles can be inserted into the tissue mass. The FNA sample obtained from the tissue mass may be acquired by one or more passages of the needle across the tissue mass. In some cases, the FNA sample can comprise less than about 6×10⁶, 5×10⁶, 4×10⁶, 3×10⁶, 2×10⁶, 1×10⁶ cells or less. The needle can be guided to the tissue mass by ultrasound or other imaging device. The needle can be hollow to permit recovery of the FNA sample through the needle by aspiration or vacuum or other suction techniques.

Samples obtained using methods disclosed herein, such as an FNA sample, may comprise a small sample volume. A sample volume may be less than about 500 microliters (uL), 400 uL, 300 uL, 200 uL, 100 uL, 75 uL, 50 uL, 25 uL, 20 uL, 15 uL, 10 uL, 5 uL, 1 uL, 0.5 uL, 0.1 uL, 0.01 uL or less. The sample volume may be less than about 1 uL. The sample volume may be less than about 5 uL. The sample volume may be less than about 10 uL. The sample volume may be less than about 20 uL. The sample volume may be between about 1 uL and about 10 uL. The sample volume may be between about 10 uL and about 25 uL.

Samples obtained using methods disclosed herein, such as an FNA sample, may comprise small sample weights. The sample weight, such as a tissue weight, may be less than about 100 milligrams (mg), 75 mg, 50 mg, 25 mg, 20 mg, 15 mg, 10 mg, 9 mg, 8 mg, 7 mg, 6 mg, 5 mg, 4 mg, 3 mg, 2 mg, 1 mg, 0.5 mg, 0.1 mg or less. The sample weight may be less than about 20 mg. The sample weight may be less than about 10 mg. The sample weight may be less than about 5 mg. The sample weight may be between about 5 mg and about 20 mg. The sample weight may be between about 1 mg and about 5 ng.

Samples obtained using methods disclosed herein, such as FNA, may comprise small numbers of cells. The number of cells of a single sample may be less than about 10×10⁶, 5.5×10⁶, 5×10⁶, 4.5×10⁶, 4×10⁶, 3.5×10⁶, 3×10⁶, 2.5×10⁶, 2×10⁶, 1.5×10⁶, 1×10⁶, 0.5×10⁶, 0.2×10⁶, 0.1×10⁶ cells or less. The number of cells of a single sample may be less than about 5×10⁶ cells. The number of cells of a single sample may be less than about 4×10⁶ cells. The number of cells of a single sample may be less than about 3×10⁶ cells. The number of cells of a single sample may be less than about 2×10⁶ cells. The number of cells of a single sample may be between about 1×10⁶ and about 5×10⁶ cells. The number of cells of a single sample may be between about 1×10⁶ and about 10×10⁶ cells.

Samples obtained using methods disclosed herein, such as FNA, may comprise small amounts of deoxyribonucleic acid (DNA) or ribonucleic acid (RNA). The amount of DNA or RNA in an individual sample may be less than about 500 nanograms (ng), 400 ng, 300 ng, 200 ng, 100 ng, 75 ng, 50 ng, 45 ng, 40 ng, 35 ng, 30 ng, 25 ng, 20 ng, 15 ng, 10 ng, 5 ng, 1 ng, 0.5 ng, 0.1 ng, or less. The amount of DNA or RNA may be less than about 40 ng. The amount of DNA or RNA may be less than about 25 ng. The amount of DNA or RNA may be less than about 15 ng. The amount of DNA or RNA may be between about 1 ng and about 25 ng. The amount of DNA or RNA may be between about 5 ng and about 50 ng.

RNA yield or RNA amount of a sample can be measured in nanogram to microgram amounts. An example of an apparatus that can be used to measure nucleic acid yield in the laboratory is a NANODROP® spectrophotometer, QUBIT® fluorometer, or QUANTUS™ fluorometer. The accuracy of a NANODROP® measurement may decrease significantly with very low RNA concentration. Quality of data obtained from the methods described herein can be dependent on RNA quantity. Meaningful gene expression or sequence variant data or others can be generated from samples having a low or un-measurable RNA concentration as measured by NANODROP®. In some cases, gene expression or sequence variant data or others can be generated from a sample having an unmeasurable RNA concentration.

The methods as described herein can be performed using samples with low quantity or quality of polynucleotides, such as DNA or RNA. A sample with low quantity or quality of RNA can be for example a degraded or partially degraded tissue sample. A sample with low quantity or quality of RNA may be a fine needle aspirate (FNA) sample. The RNA quality of a sample can be measured by a calculated RNA Integrity Number (RIN) value. The RIN value is an algorithm for assigning integrity values to RNA measurements. The algorithm can assign a 1 to 10 RIN value, where an RIN value of 10 can be completely intact RNA. A sample as described herein that comprises RNA can have an RIN value of about 9.0, 8.0, 7.0, 6.0, 5.0, 4.0, 3.0, 2.0, 1.0 or less. In some cases, a sample comprising RNA can have an RIN value equal or less than about 8.0. In some cases, a sample comprising RNA can have an RIN value equal or less than about 6.0. In some cases, a sample comprising RNA can have an RIN value equal or less than about 4.0. In some cases, a sample can have an RIN value of less than about 2.0.

A sample, such as an FNA sample, may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. A medical professional may obtain a sample from a subject for testing or refer the subject to a testing center or laboratory for the submission of the sample. The medical professional may indicate to the testing center or laboratory the appropriate test or assay to perform on the sample, such as methods of the present disclosure including determining gene sequence data, gene expression levels, sequence variant data, or any combination thereof.

In some cases, a medical professional need not be involved in the initial diagnosis of a disease or the initial sample acquisition. An individual, such as the subject, may alternatively obtain a sample through the use of an over the counter kit. The kit may contain collection unit or device for obtaining the sample as described herein, a storage unit for storing the sample ahead of sample analysis, and instructions for use of the kit.

A sample can be obtained a) pre-operatively, b) post-operatively, c) after a cancer diagnosis, d) during routine screening following remission or cure of disease, e) when a subject is suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

### Cytological Analysis

The methods as described herein may include cytological analysis of samples. The methods may use cytological analysis to examine the samples for malignancy. For example, the sample may be determined to be not benign based on cytological analysis. Examples of cytological analysis include cell staining techniques and/or microscope examination performed by any number of methods and suitable reagents including but not limited to: eosin-azure (EA) stains, hematoxylin stains, CYTO-STAIN™, papanicolaou stain, eosin, nissl stain, toluidine blue, silver stain, azocarmine stain, neutral red, or janus green. More than one stain can be used in combination with other stains. In some cases, cells are not stained at all. Cells can be fixed and/or permeabilized with for example methanol, ethanol, glutaraldehyde or formaldehyde prior to or during the staining procedure. In some cases, the cells may not be fixed. Staining procedures can also be utilized to measure the nucleic acid content of a sample, for example with ethidium bromide, hematoxylin, nissl stain or any other nucleic acid stain.

Microscope examination of cells in a sample can include smearing cells onto a slide by standard methods for cytological examination. Liquid based cytology (LBC) methods may be utilized. In some cases, LBC methods provide for an improved approach of cytology slide preparation, more homogenous samples, increased sensitivity and specificity, or improved efficiency of handling of samples, or any combination thereof. In LBC methods, samples can be transferred from the subject to a container or vial containing a LBC preparation solution such as for example CYTYC THINPREP®, SUREPATH™, or MONOPREP® or any other LBC preparation solution. Additionally, the sample may be rinsed from the collection device with LBC preparation solution into the container or vial to ensure substantially quantitative transfer of the sample. The solution containing the sample in LBC preparation solution may then be stored and/or processed by a machine or by one skilled in the art to produce a layer of cells on a glass slide. The sample may further be stained and examined under the microscope in the same way as a conventional cytological preparation.

Samples can be analyzed by immuno-histochemical staining. Immuno-histochemical staining can provide analysis of the presence, location, and distribution of specific molecules or antigens by use of antibodies in a sample (e.g. cells or tissues). Antigens can be small molecules, proteins, peptides, nucleic acids or any other molecule capable of being specifically recognized by an antibody. Samples may be analyzed by immuno-histochemical methods with or without a prior fixing and/or permeabilization step. In some cases, the antigen of interest may be detected by contacting the sample with an antibody specific for the antigen and then non-specific binding may be removed by one or more washes. The specifically bound antibodies may then be detected by an antibody detection reagent such as for example a labeled secondary antibody, or a labeled avidin/streptavidin. The antigen specific antibody can be labeled directly. Suitable labels for immunohistochemistry include but are not limited to fluorophores such as fluorescein and rhodamine, enzymes such as alkaline phosphatase and horse radish peroxidase, or radionuclides such as ³²P and ¹²⁵I. Gene product markers that may be detected by immuno-histochemical staining include but are not limited to Her2/Neu, Ras, Rho, EGFR, VEGFR, UbcH10, RET/PTC1, cytokeratin 20, calcitonin, GAL-3, thyroid peroxidase, or thyroglobulin.

Metrics associated with classifying a tissue sample as disclosed herein, such as sequences corresponding to mRNA transcripts, mitochondrial transcripts, and/or chromosomal loss of heterozygosity, need not be a characteristic of every cell of a sample found to comprise the tissue classification. Thus, the methods disclosed herein can be useful for classifying a tissue sample, e.g. as invasive or metastatic, within a tissue where less than all cells within the sample exhibit a complete pattern of the gene expression levels or sequence variant data, or other data indicative of tissue classification. The gene expression levels, sequence variant data, or others may be either completely present, partially present, or absent within affected cells, as well as unaffected cells of the sample. The gene expression levels, sequence variant data, or others may be present in variable amounts within affected cells. The gene expression levels, sequence variant data, or others may be present in variable amounts within unaffected cells. In some cases, the gene expression levels of a first set of genes or the presence of one or more sequence variants in a second set of genes that correlates with a risk of being invasive or metastatic can be positively detected. In some instances, positive detection can occur in at least 70%, 75%, 80%, 85%, 90%, 95%, or 100% of cells drawn from a sample. In some cases, the gene expression levels of a first set of genes or the presence of one or more sequence variants in a second set of genes can be absent. In some instances, absence of detection can occur in at least 70%, 75%, 80%, 85%, 90%, 95%, or 100% of cells of a corresponding normal or benign, non-disease sample.

Routine cytological or other assays may indicate a sample as negative (without disease), diagnostic (positive diagnosis for disease, such as cancer), ambiguous or suspicious (e.g., indeterminate) (suggestive of the presence of a disease, such as cancer), or non-diagnostic (providing inadequate information concerning the presence or absence of disease).

The methods as described herein may confirm results from the routine cytological assessments or may provide an original assessment similar to a routine cytological assessment in the absence of one. The methods as described herein may classify a sample as invasive or metastatic, including samples found to be ambiguous, suspicious, or indeterminate. The methods may further stratify samples, such as samples known to be invasive or metastatic, into low risk and medium-to-high risk of invasion or metastases, including samples found to be ambiguous, suspicious, or indeterminate. For example, a high risk of being invasive may comprise having an extent of invasion that is at least one of extensive lympho-vascular invasion, focal extra-thyroidal invasion, or extensive extra-thyroidal invasion.

The method provided herein may comprise reactions for assaying nucleic acids. For example, nucleic acids may be assayed using array hybridization or sequencing. Array hybridization or sequencing may be used identify to presence of a gene expression products or determine a gene expression level. Determining gene expression product levels may comprise performing one or more of the following: additional cytological assays, assays for specific proteins or enzyme activities, assays for specific expression products including protein or RNA or specific RNA splice variants, in situ hybridization, whole or partial genome expression analysis, microarray hybridization assays, SAGE, enzyme linked immuno-absorbance assays, mass-spectrometry, immuno-histochemistry, blotting, sequencing, RNA sequencing, DNA sequencing (e.g., sequencing of cDNA obtained from RNA); Next-Gen sequencing, nanopore sequencing, pyrosequencing, or Nanostring sequencing. Gene expression product levels may be normalized to an internal standard such as total mRNA or the expression level of a particular gene.

For example, microarray technology can be used to measure the relative activity of previously identified target genes and other expressed sequences. Sequence based techniques, like serial analysis of gene expression (SAGE, SuperSAGE) are also used for gene expression profiling. SuperSAGE is especially accurate and can measure any active gene, not just a predefined set. In an RNA, mRNA or gene expression profiling microarray, the expression levels of thousands of genes can be simultaneously monitored to study the effects of certain treatments, diseases, and developmental stages on gene expression. For example, microarray-based gene expression profiling can be used to characterize gene signatures of a genetic disorder disclosed herein, or different cancer types, subtypes of a cancer, and/or cancer stages.

Suitable reagents for conducting array hybridization, nucleic acid sequencing, nucleic acid amplification or other amplification reactions include, but are not limited to, DNA polymerases, markers such as forward and reverse primers, deoxynucleotide triphosphates (dNTPs), and one or more buffers. Such reagents can include a primer that is selected for a given sequence of interest, such as the one or more genes of the first set of genes and/or second set of genes.

In such amplification reactions, one primer of a primer pair can be a forward primer complementary to a sequence of a target polynucleotide molecule (e.g. the one or more genes of the first or second sets) and one primer of a primer pair can be a reverse primer complementary to a second sequence of the target polynucleotide molecule and a target locus can reside between the first sequence and the second sequence.

The length of the forward primer and the reverse primer can depend on the sequence of the target polynucleotide (e.g., the one or more genes of the first or second sets) and the target locus. In some cases, a primer can be greater than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or about 100 nucleotides in length. As an alternative, a primer can be less than about 100, 95, 90, 85, 80, 75, 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or about nucleotides in length. In some cases, a primer can be about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, about 20 to about 60, about 20 to about 80, or about 20 to about 100 nucleotides in length.

Primers can be designed according to known parameters for avoiding secondary structures and self-hybridization, such as primer dimer pairs. Different primer pairs can anneal and melt at about the same temperatures, for example, within 1° C., 2° C., 3° C., 4° C., 5° C., 6° C., 7° C., 8° C., 9° C. or 10° C. of another primer pair.

The target locus can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends of the plurality of template polynucleotides.

Markers (i.e., primers) for the methods described can be one or more of the same primer. In some instances, the markers can be one or more different primers such as about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more different primers. In such examples, each primer of the one or more primers can comprise a different target or template specific region or sequence, such as the one or more genes of the first or second sets.

One or more primers can comprise a fixed panel of primers. The one or more primers can comprise at least one or more custom primers. The one or more primers can comprise at least one or more control primers. The one or more primers can comprise at least one or more housekeeping gene primers. In some instances, the one or more custom primers anneal to a target specific region or complements thereof. The one or more primers can be designed to amplify or to perform primer extension, reverse transcription, linear extension, non-exponential amplification, exponential amplification, PCR, or any other amplification method of one or more target or template polynucleotides.

Primers can incorporate additional features that allow for the detection or immobilization of the primer but do not alter a basic property of the primer (e.g., acting as a point of initiation of DNA synthesis). For example, primers can comprise a nucleic acid sequence at the 5′ end which does not hybridize to a target nucleic acid, but which facilitates cloning or further amplification, or sequencing of an amplified product. For example, the sequence can comprise a primer binding site, such as a PCR priming sequence, a sample barcode sequence, or a universal primer binding site or others.

A universal primer binding site or sequence can attach a universal primer to a polynucleotide and/or amplicon. Universal primers can include-47F (M13F), alfaMF, AOX3′, AOX5′, BGHr, CMV-30, CMV-50, CVMf, LACrmt, lamgda gt10F, lambda gt10R, lambda gt11F, lambda gt11R, M13 rev, M13Forward (−20), M13Reverse, male, p10SEQPpQE, pA-120, pet4, pGAP Forward, pGLRVpr3, pGLpr2R, pKLAC14, pQEFS, pQERS, pucU1, pucU2, reversA, seqIREStam, seqIRESzpet, seqori, seqPCR, seqpIRES-, seqpIRES+, seqpSecTag, seqpSecTag+, seqretro+PSI, SP6, T3-prom, T7-prom, and T7-termInv. As used herein, attach can refer to both or either covalent interactions and noncovalent interactions. Attachment of the universal primer to the universal primer binding site may be used for amplification, detection, and/or sequencing of the polynucleotide and/or amplicon.

### Trained Algorithm

The trained algorithm of the present disclosure can be trained using a set of samples, such as a sample cohort. The sample cohort can comprise about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise about 100 independent samples. The sample cohort can comprise about 200 independent samples. The sample cohort can comprise between about 100 and about 700 independent samples. The independent samples can be from subjects having been diagnosed with a disease, such as cancer, from healthy subjects, or any combination thereof.

The sample cohort can comprise samples from about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from about 100 different individuals. The sample cohort can comprise samples from about 200 different individuals. The different individuals can be individuals having been diagnosed with a disease, such as cancer, health individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations include, but are not limited to, test centers, medical facilities, medical offices, post office addresses, cities, counties, states, nations, or continents. In some cases, a classifier that is trained using sample cohorts from the United States may need to be re-trained for use on sample cohorts from other geographical regions (e.g., India, Asia, Europe, Africa, etc.).

The trained algorithm may comprise one or more classifiers that identify the presence of a gene or a plurality of genes. For example, classifier may identify the presence or absence of a BRAF gene. The one or more classifiers may identify the expression of a plurality of different genes associated with various gene signatures. The one or more classifiers may identify the expression of a plurality of different variants or genetic aberrations. The one or more classifiers may identify the presence of one or more gene signatures in a subject. The one or more classifiers may use one or more gene signatures to make a classification. The gene signatures may be a set of genes that are associated with a particular attribute (e.g., phenotypical attribute, disease state) or a process or activity. For example, the gene signatures may be a set of genes associated with specific metabolic processes. The gene signatures may be related to cancer hallmarks such as endothelial to mesenchymal transition, tumor microenvironment, immune-oncology, metabolism, and treatment sensitivity. The gene signatures may be related to cancer. The gene signatures may be related to follicular thyroid cancer. The gene signatures may comprise genes associated with BRAF. The gene signatures may comprise genes associated with RAS. The gene signature may comprise genes associated with androgen receptors. The gene signature may be associated with epithelial mesenchymal transition (EMT). The gene signature may be associated with risk of recurrence (ROR). The gene signatures may be a set of genes that were identified and identified in a literature reference. The gene signatures may be a set of variants that were identified and identified in a literature reference. The gene signature may comprise one or more genes. The gene signatures may comprise at least 1, 2, 3, 4, 5, 6, 7, 8, 9, 10, 11, 12, 13, 14, 15, 16, 17, 19, 20, 21, 22, 23, 24, 25, 26, 27, 28, 29, 30, 40, 50, 60, 70, 80, 90, 100, 120, 140, 160, 180, 200, 220, 240, 260, 280, 300, 400, 500, 600,700, 800, 900, 1000, 2000, 3000, 4000, 5000, 6000, 7000, 8000, 9000, 10000, or more genes. The gene signatures may comprise no more than 1, 2, 3, 4, 5, 6, 7, 8, 9, 10, 11, 12, 13, 14, 15, 16, 17, 19, 20, 21, 22, 23, 24, 25, 26, 27, 28, 29, 30, 40, 50, 60, 70, 80, 90, 100, 120, 140, 160, 180, 200, 220, 240, 260, 280, 300, 400, 500, 600,700, 800, 900, 1000, 2000, 3000, 4000, 5000, 6000, 7000, 8000, 9000, 10000, or less genes.

The one or more classifiers may identify the expression of genes that are differentially expressed between different sample types. For example, the one or more classifiers may identify the expression of genes that are differentially expressed between samples with a low risk of invasion and samples with a high risk of invasion. The one or more classifiers may be an ensemble classifier.

The trained algorithm may be a machine learning algorithm. The machine learning algorithm may comprise a Penalized Generalized linear model (PGLM), a hierarchical Penalized Generalized linear model, Random Forest (RF) model, Support Vector Machine (SVM) model and SVM-radial model, or an ensemble model using a combination of models.

In some embodiments, the specificity of the present method is at least 60%, 65%, 70%, 75%, 80%, 85%, 86%, 87%, 88%, 89%, 90%, 91%, 92%, 93%, 94%, 95%, 96%, 97%, 98%, 99%, or more.

In some embodiments, the sensitivity of the present method is at least 70%, 75%, 80%, 85%, 86%, 87%, 88%, 89%, 90%, 91%, 92%, 93%, 94%, 95%, 96%, 97%, 98%, 99%, or more.

In some embodiments, the specificity is greater than or equal to 60%. The negative predictive value (NPV) is greater than or equal to 95%. In some embodiments, the NPV is at least 95%, 95.5%, 96%, 96.5%, 97%, 97.5%, 98%, 98.5%, 99%, 99.5% or more.

Sensitivity typically refers to TP/(TP+FN), where TP is true positive and FN is false negative. Number of Continued Indeterminate results divided by the total number of malignant results based on adjudicated histopathology diagnosis. Specificity typically refers to TN/(TN+FP), where TN is true negative and FP is false positive. The number of actual benign results is divided by the total number of benign results based on adjudicated histopathology diagnosis. Positive Predictive Value (PPV) may be determined by: TP/(TP+FP). Negative Predictive Value (NPV) may be determined by TN/(TN+FN).

A biological sample may be identified as invasive, non-invasive, metastatic, or non-metastatic with an accuracy of greater than 75%, 80%, 85%, 90%, 95%, 99% or more. In some embodiments, the biological sample is identified as invasive, non-invasive, metastatic, or non-metastatic with a sensitivity of greater than 90%. In some embodiments, the biological sample is identified as invasive, non-invasive, metastatic, or non-metastatic with a specificity of greater than 60%. In some embodiments, the biological sample is identified as invasive, non-invasive, metastatic, or non-metastatic with a sensitivity of greater than 90% and a specificity of greater than 60%. In some embodiments, the accuracy is calculated using a trained algorithm.

Results of the expression analysis of the subject methods may provide a statistical confidence level that a given diagnosis is correct. In some embodiments, such statistical confidence level is above 85%, 90%, 91%, 92%, 93%, 94%, 95%, 96%, 97%, 98%, 99% or 99.5%.

A trained algorithm may produce a unique output each time it is run. For example, using a different sample or plurality of samples with the same classifier can produce a unique output each time the classifier is run. Using the same sample or plurality of samples with the same classifier can produce a unique output each time the classifier is run. Using the same samples to train a classifier more than one time, may result in unique outputs each time the classifier is run.

Sequence information refers herein to mRNA or gene expression, mitochondrial transcripts, genetic variants and/or fusion transcripts. In some embodiments, sequence information is gene expression level and/or sequence variants (i.e., genetic variants). For example, sequences obtained from a sample can be compared to a reference set to determine the presence of one or more sequence variants in a sample. As another example, gene expression levels of one or more genes from a sample can be processed relative to expression levels of a reference set of genes to determine the presence of differential gene expression of said one or more genes. A reference set can comprise one or more housekeeping genes. A reference set can comprise known sequence variants and/or expression levels of genes known to be associated with a particular disease or known to be associated with a non-disease state.

Characteristics of a sample (e.g., sequence information as defined above) can be analyzed using an algorithm that comprises one or more classifiers and which is trained using one or more an annotated reference sets. The identification can be performed by the classifier. More than one characteristic of a sample can be combined to generate classification of tissue sample. For example, sequence information corresponding to mRNA expression and mitochondrial transcripts can be combined and a classification can be generated from the combined data. The cytological classification of a sample be analyzed by the algorithm. The characteristic may be a clinical covariate. For example, the characteristics may be a gender, cytology, or cohort. The cohort from which a sample is derived may be used characteristic of a sample to improve the predictive metrics. The combining can be performed by the classifier. In another example, sequences obtained from a sample can be compared to a reference set to determine the presence of one or more sequence variants in a sample. In some cases, gene expression levels of one or more genes from a sample can be processed relative to expression levels of a reference set of genes that are used to train one or more classifiers to determine the presence of differential gene expression of one or more genes. A reference set can comprise one or more housekeeping genes. The reference set can comprise known sequence variants and/or expression levels of genes known to be associated with a particular disease or known to be associated with a non-disease state.

The one or more reference sets of genes may be grouped into gene signatures pertaining to, or derived from literature studies. The gene signatures may be a set of genes that are associated with a particular attribute (e.g., phenotypical attribute, disease state) or a process or activity. For example, studies on differing aspect of cell biology may be analyzed and annotated for gene expression, or changes to gene expression. The signatures may be related to a variety of cell activities or mechanism, for example genes relating to drug metabolism, genes that affect or are related to cancer hallmarks such as endothelial to mesenchymal transition, tumor microenvironment, immune-oncology, metabolism, and treatment sensitivity. The signatures may be used to train the one or more classifiers. The signatures may be related to expression of genes that are differentially expressed between different sample types. For example, the one or more classifiers may identify the expression of genes that are differentially expressed between samples with a low risk of invasion and samples with a high risk of invasion. The gene signatures may be related to expression of genetic aberrations that are related to cancer hallmarks such as endothelial to mesenchymal transition, tumor microenvironment, immune-oncology, metabolism, and treatment sensitivity. The gene signatures may be related to expression of genetic variations that are related to cancer hallmarks such as endothelial to mesenchymal transition, tumor microenvironment, immune-oncology, metabolism, and treatment sensitivity. The gene signatures may comprise genes associated with BRAF. The gene signatures may comprise genes associated with RAS. The gene signature may comprise genes associated with androgen receptors. The gene signature may be associated with epithelial mesenchymal transition (EMT). The gene signature may be associated with risk of recurrence (ROR). The gene signatures may comprise at least 1, 2, 3, 4, 5, 6, 7, 8, 9, 10, 11, 12, 13, 14, 15, 16, 17, 19, 20, 21, 22, 23, 24, 25, 26, 27, 28, 29, 30, 40, 50, 60, 70, 80, 90, 100, 120, 140, 160, 180, 200, 220, 240, 260, 280, 300, 400, 500, 600,700, 800, 900, 1000, 2000, 3000, 4000, 5000, 6000, 7000, 8000, 9000, 10000, or more genes. The gene signatures may comprise no more than 1, 2, 3, 4, 5, 6, 7, 8, 9, 10, 11, 12, 13, 14, 15, 16, 17, 19, 20, 21, 22, 23, 24, 25, 26, 27, 28, 29, 30, 40, 50, 60, 70, 80, 90, 100, 120, 140, 160, 180, 200, 220, 240, 260, 280, 300, 400, 500, 600,700, 800, 900, 1000, 2000, 3000, 4000, 5000, 6000, 7000, 8000, 9000, 10000, or less genes.

Classifiers of a trained algorithm can perform processing, combining, statistical evaluation, or further analysis of results, or any combination thereof. Separate reference sets may be provided for different features. For example, sequence variant data may be processed relative to a sequence variant data reference set. A gene expression level data may be processed relative to a gene expression level reference set. In some cases, multiple feature spaces may be processed with respect to the same reference set.

In some cases, sequence variants of a particular gene may or may not affect the gene expression level of that same gene. A sequence variant of a particular gene may affect the gene expression level of one or more different genes that may be located adjacent to and distal from the particular gene with the sequence variant. The presence of one or more sequence variants can have downstream effects on one or more genes. A sequence variant of a particular gene may perturb one or more signalling pathways, may cause ribonucleic acid (RNA) transcriptional regulation changes, may cause amplification of deoxyribonucleic acid (DNA), may cause multiple transcript copies to be produced, may cause excessive protein to be produced, may cause single base pairs, multi-base pairs, partial genes or one or more genes to be removed from the sequence.

Data from the methods described, such as gene expression levels or sequence variant data can be further analyzed using feature selection techniques such as filters which can assess the relevance of specific features by looking at the intrinsic properties of the data, wrappers which embed the model hypothesis within a feature subset search, or embedded protocols in which the search for an optimal set of features is built into a classifier algorithm. The trained algorithm or classifier may comprise or use one or more features (e.g., gene expression or gene expression levels, sequence variant, gene signature, clinical attribute) to output a classification. For example, the trained algorithm or classifier may comprise or use at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more features. For example, the trained algorithm or classifier may comprise or use at least 5 features. For example, the trained algorithm or classifier may comprise or use at least 10 features. For example, the trained algorithm or classifier may comprise or use at least 20 features. For example, the trained algorithm or classifier may comprise or use no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less features. For example, the trained algorithm or classifier may comprise or use no more than 5 features. For example, the trained algorithm or classifier may comprise or use no more than 10 features. For example, the trained algorithm or classifier may comprise or use no more than 20 features. The use of a larger number of features may increase the performance of classifier or algorithm at classifying a tissue. However, the use of smaller number of features may allow for a similar performance as an algorithm with a larger number of features, with a minimal marginal improvement with the addition of additional features. For example, an algorithm designed by the methods described herein may use only five or fewer features and a have a similar performance to algorithms with 20 or more features. The methods described herein allow for algorithms with varying numbers of features that can be modulated based on desired performance metrics or amount of data pertained to features that is available. For example, algorithms with fewer features may require less data to be collected on a given subject, while still providing an accurate, specific, or sensitive classification.

Filters useful in the methods of the present disclosure can include, for example, (1) parametric methods such as the use of two sample t-tests, analysis of variance (ANOVA) analyses, Bayesian frameworks, or Gamma distribution models (2) model free methods such as the use of Wilcoxon rank sum tests, between-within class sum of squares tests, rank products methods, random permutation methods, or threshold number of misclassification (TNoM) which involves setting a threshold point for fold-change differences in expression between two datasets and then detecting the threshold point in each gene that minimizes the number of mis-classifications or (3) multivariate methods such as bivariate methods, correlation based feature selection methods (CFS), minimum redundancy maximum relevance methods (MRMR), Markov blanket filter methods, and uncorrelated shrunken centroid methods. Wrappers useful in the methods of the present disclosure can include sequential search methods, genetic algorithms, or estimation of distribution algorithms. Embedded protocols can include random forest algorithms, weight vector of support vector machine algorithms, or weights of logistic regression algorithms.

Statistical evaluation of the results obtained from the methods described herein can provide a quantitative value or values indicative of one or more of the following: the classification of the tissue sample; the likelihood of diagnostic accuracy; the likelihood of disease, such as cancer; the likelihood of a particular disease, such as a tissue-specific cancer, for example, thyroid cancer; and the likelihood of the success of a particular therapeutic intervention. Thus a medical professional, who may not be trained in genetics or molecular biology, need not understand gene expression level or sequence variant data results. Rather, data can be presented directly to the medical professional in its most useful form to guide care or treatment of the subject. Statistical evaluation, combination of separate data results, and reporting useful results can be performed by the trained algorithm. Statistical evaluation of results can be performed using a number of methods including, but not limited to: the students T test, the two sided T test, Pearson rank sum analysis, hidden Markov model analysis, analysis of q-q plots, principal component analysis, one way analysis of variance (ANOVA), two way ANOVA, and the like. Statistical evaluation can be performed by the trained algorithm.

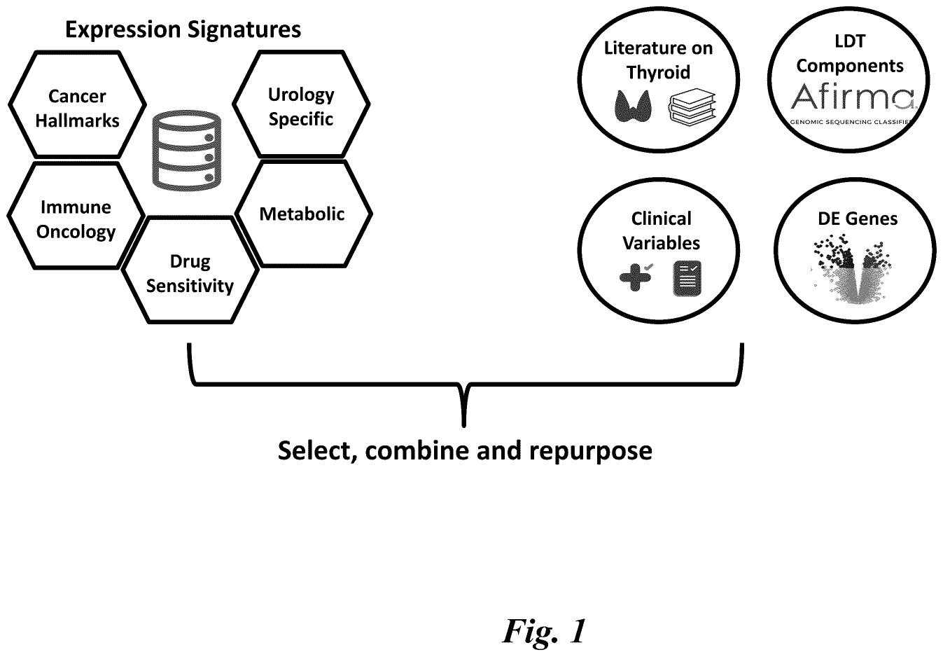

FIG. 1 shows an overview of the different features and methods that can be used to rule out high risk of invasion or metastasis. Different features such as expression signatures (e.g., cancer hallmark genes, genes associated with immune oncology, genes specific to urology, genes associated with metabolism, or specific metabolic pathways, genes associated with sensitivity to drugs) along with literature derived information data from other laboratory developed tests, differentially expressed genes, and clinical variables can be used for creation of a model or algorithm. These different features can be combined and selected to generate an algorithm that can classify a sample.

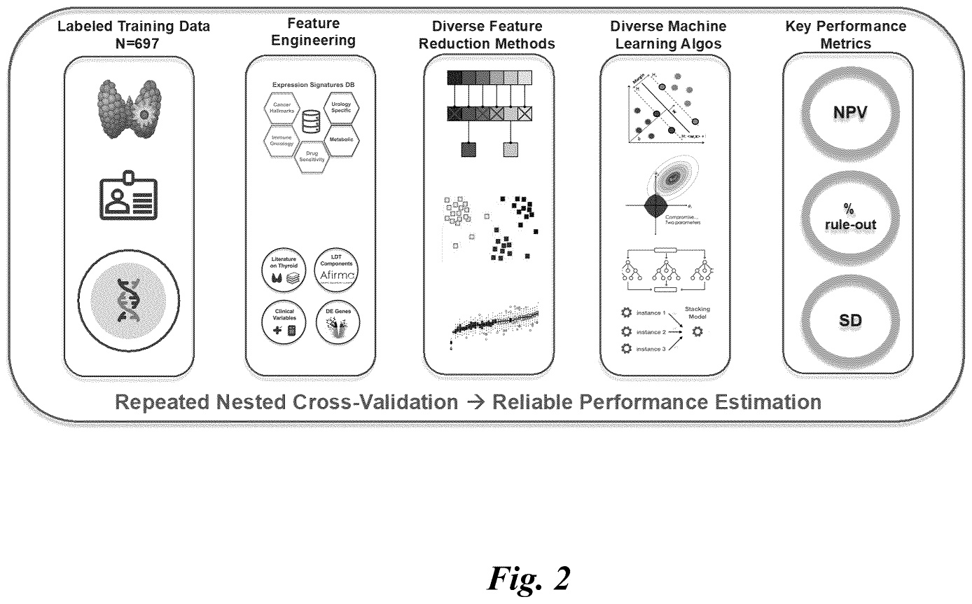

FIG. 2 shows an overview of the method and general step and parameters used to develop multiple different machine learning models (e.g., greater than 400 machine learning models). Starting with initial training data set, a set of patients that demonstrate specific attributes (e.g., nodes of a certain size or at a certain location) can be labelled or a categorized). Feature engineering can be performed such as features shown in FIG. 1 to generate a number of feature that may be relevant or helpful in classifying a sample The models can be created using feature reduction methods and different machine learning algorithms, which can generate specific classifier that use a smaller number of features that are identified as relevant or important for classification. These models can be subjected to repeated nested cross-validation, and also verified using additional clinical cohorts, in order to find those models with reliable performance metrics.

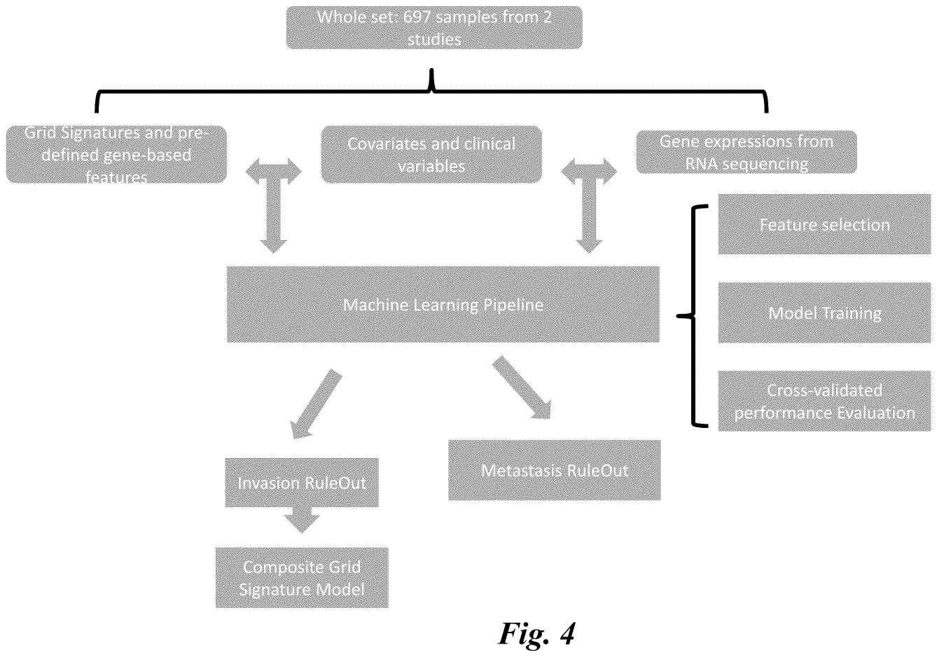

As shown in FIG. 4, the method can be used to build mRNA expression-based risk signatures with a high negative predictive value for ruling out tumor invasion and metastases. As shown, a set of samples (e.g., 697 samples from two separate data sets) can be used as a training set to train a model. GRID signatures and predefined gene-based features, covariates and clinical variables, and gene expression can be used as potential features in the model. These features can then be inputted into the machine learning pipeline, which will perform feature selection, train the model, and perform cross-validation and performance evaluation. Based on different features selection methods and machine learning algorithms, a large number of models can be generated and then validated. These models can be selected for those that allow for a rule out of high risk samples.

### Diseases

A disease, as disclosed herein, can include thyroid cancer. Thyroid cancer can include any subtype of thyroid cancer, including but not limited to, any malignancy of the thyroid gland such as papillary thyroid cancer (PTC), follicular thyroid cancer (FTC), follicular variant of papillary thyroid carcinoma (FVPTC), medullary thyroid carcinoma (MTC), follicular carcinoma (FC), Hurthle cell carcinoma (HC), and/or anaplastic thyroid cancer (ATC). In some cases, the thyroid cancer can be differentiated. In some cases, the thyroid cancer can be undifferentiated.

A thyroid tissue sample can be classified using the methods of the present disclosure as comprising one or more benign or malignant tissue types (e.g. a cancer subtype), including but not limited to follicular adenoma (FA), nodular hyperplasia (NHP), lymphocytic thyroiditis (LCT), and Hurthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), and anaplastic thyroid carcinoma (ATC), renal carcinoma (RCC), breast carcinoma (BCA), melanoma (MMN), B cell lymphoma (BCL), or parathyroid (PTA).

### Monitoring of Subjects or Therapeutic Interventions Via Molecular Profiling

In the methods of the present disclosure, a subject may be monitored. For example, a subject may be diagnosed with cancer. This initial diagnosis may or may not involve the use of methods disclosed herein. The subject may be prescribed a therapeutic intervention such as a thyroidectomy for a subject suspected of having thyroid cancer. The results of the therapeutic intervention may be monitored on an ongoing basis by methods disclosed herein to detect the efficacy of the therapeutic intervention. In another example, a subject may be diagnosed with a benign tumor or a precancerous lesion or nodule, and the tumor, nodule, or lesion may be monitored on an ongoing basis by methods disclosed herein to detect any changes in the state of the tumor or lesion.

### Methods of Therapeutic Interventions

In the methods of the present disclosure, a subject may be provided a therapeutic intervention. For example, a subject may be diagnosed as having an invasive, non-invasive, metastatic or non-metastatic malignancy. Based on the classification of a malignancy, the different therapeutic intervention may be provided. For example, the subject may be prescribed a therapeutic intervention such as a thyroidectomy for a subject having a high risk of an invasive or metastatic thyroid cancer. In another example, a subject may be diagnosed with a non-invasive tumor or non-metastatic tumor and may receive a recommendation to not receive a surgical intervention. In this way, the methods may allow for a personalized treatment and may reduce unnecessary surgeries that could otherwise lead to surgical complications or post-operative loss of functions (e.g., hypothyroidism for thyroid removal).

### Computer Systems

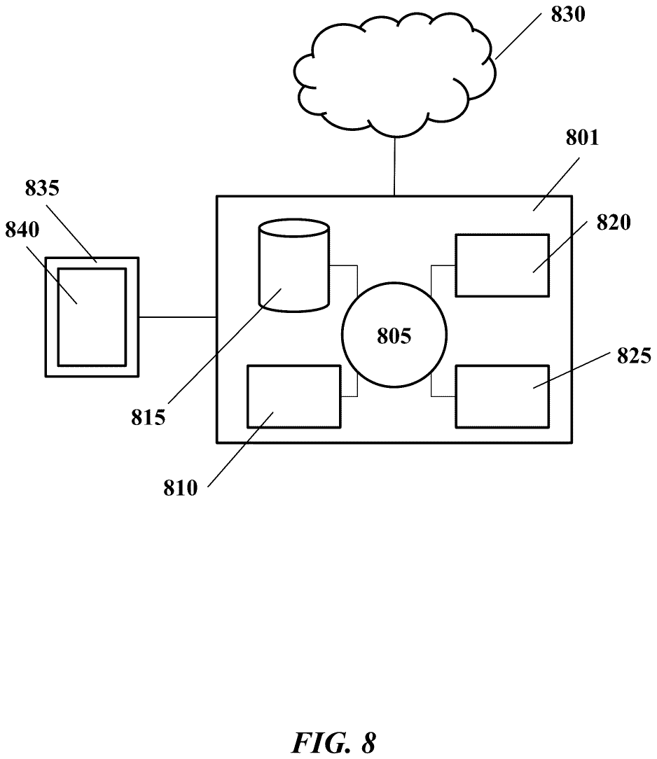

The present disclosure provides computer systems that are programmed to implement methods of the disclosure. FIG. 8 shows a computer system 801 that is programmed or otherwise configured to implement the trained algorithm for the classifier of invasive or metastatic samples. The computer system 801 can regulate various aspects of the methods of the present disclosure, such as, for example, nucleic acid sequencing methods, interpretation of nucleic acid sequencing data and analysis of cellular nucleic acids, such as RNA (e.g., mRNA), and characterization of samples from sequencing data. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. For instance, the computer system 801 can communicate with a remote computer system of a user (e.g., medical professional, or subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, results of nucleic acid sequencing, analysis of nucleic acid sequencing data, characterization of nucleic acid sequencing samples, tissue characterizations, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, initiate nucleic acid sequencing, process nucleic acid sequencing data, interpret nucleic acid sequencing results, characterize nucleic acid samples, characterize samples, etc.

### EXAMPLES

### Example 1. Parameterization of Extent of Invasion and Extent of Metastases in FNA Biopsy Samples and Training Cohorts

Histopathology reports from Afirma Genomic Sequencing Classifier (GSC) algorithm training and from thyroid cancer patients managed at an integrative endocrine surgery community care practice (total 697 and ~50% from each) were reviewed for invasion and metastases. An integer from 0-3 was assigned based on the extent of invasion (ranging from none to extensive extra-thyroidal) or extent of metastases (ranging from none to lateral node metastases), as shown in FIG. 3. For tumor invasion, if the pathology reported vascular invasion of ≥4 blood vessels (or described extensive vascular invasion) or there was extrathyroidal extension, the sample was labeled as high risk. Otherwise, the sample was labeled as low risk for tumor invasion. For LNM, if the pathology reported >2 mm central lymph node deposits and/or >40% of the central nodes resected as malignant, or if there was lateral lymph node thyroid cancer involvement, the sample was labeled as high risk for LNM. Otherwise, the sample was labeled as low risk. Samples could be high risk for one category and low risk for another. Invasion labels of 0/1 corresponded to a low risk and were regarded as a negative class. Invasion labels of 2/3 corresponded to a high risk of invasion and were regarded as a positive class. Metastasis labels of 0/1 corresponded to a low risk and were regarded as a negative class. Metastasis labels of 2/3 corresponding to a high risk of invasion and were regarded as a positive class. This analyzed and labeled patient data set was then used to train the models.

Tables 1A-1C is a summary of the cohort training set.

TABLE 1A-1C
Clinicogenomic characteristics of the training cohorts.

| | Training | | | | | |
|---|---|---|---|---|---|---|
| | Training | | Training | | Training | |
| | cohort 1 | | cohort 2 | | combined | |
| Total n | 379 | | 318 | | 697 | |
| Age (median IQR) | 52 | [39-63] | 49 | [37-59] | 51 | [38-60] |

Gender

| | | | | | | |
|---|---|---|---|---|---|---|
| Male | 91 | (24%) | 61 | (19.2%) | 152 | (21.8%) |
| Female | 288 | (76%) | 257 | (80.8%) | 545 | (78.2%) |

Bethesda cytology category

| | | | | | | |
|---|---|---|---|---|---|---|
| III | 174 | (46%) | 79 | (24.8%) | 253 | (36.3%) |
| IV | 77 | (20.3%) | 55 | (17.3%) | 132 | (18.9%) |
| V | 71 | (18.7%) | 41 | (12.9%) | 112 | (16%) |
| VI | 57 | (15%) | 143 | (45%) | 200 | (28.7%) |

Invasion labels

| | | | | | | |
|---|---|---|---|---|---|---|
| low risk: no invasion | 308 | (81.2%) | 234 | (73.6%) | 542 | (77.7%) |
| Low risk: min invasion (<4 vessels) | 28 | (7.4%) | 57 | (17.9%) | 85 | (12.2%) |
| High risk: Extensive vascular | 34 | (9%) | 13 | (4.1%) | 47 | (6.7%) |
| invasion (>=4) | | | | | | |
| High risk: Extensive extra-thyroidal | 9 | (2.4%) | 14 | (4.4%) | 23 | (3.3%) |

Lymph node metastasis labels

| | | | | | | |
|---|---|---|---|---|---|---|
| Low risk: no nodes | 343 | (90.5%) | 215 | (67.6%) | 558 | (80%) |
| Low risk: central neck <2 mm | 16 | (4.2%) | 44 | (13.8%) | 60 | (8.6%) |
| or <40% LN involvement | | | | | | |
| High risk: central neck >2 mm | 12 | (3.2%) | 41 | (12.9%) | 53 | (7.6%) |
| or >40% LN involvement | | | | | | |
| High risk: Lateral neck nodes | 8 | (2.1%) | 18 | (5.7%) | 26 | (3.7%) |

Molecular alteration

| | | | | | | |
|---|---|---|---|---|---|---|
| BRAFV600E | 70 | (18.5%) | 166 | (52.2%) | 236 | (33.9%) |

TABLE 1B

| | Invasion Levels | | | | | |
|---|---|---|---|---|---|---|
| | | | High Risk | | | |
| | Low Risk | | 2 | 3 | | 2/3 |
| | 0 | 1 | (extensive | (extensive | | Prevalence |
| | (no | (min | vascular | extra- | | (High risk |
| Sample set | invasion) | invasion) | invasion | thyroidal) | Sum | Prevalence) |
| Training Cohort 1 | 308 | 28 | 34 | 9 | 379 | 11.3% |
| Training Cohort 2 | 234 | 57 | 13 | 14 | 318 | 8.5% |
| Sum | 542 | 85 | 47 | 23 | 697 | 10.0% |

TABLE 1C

| | Metastasis Levels | | | | | |
|---|---|---|---|---|---|---|
| | | | High Risk | | | |
| | | | 2 | | | |
| | Low Risk | | (central | 3 | | 2/3 |
| | 0 | 1 | neck >2 mm | (lateral | | Prevalence |
| | (no | (central | or >40% LN | neck | | High risk |
| Sample set | nodes) | neck <2 mm) | involvement) | nodes) | Sum | Prevalence) |
| Training cohort 1 | 343 | 16 | 12 | 8 | 379 | 5.3% |
| Training cohort 2 | 215 | 44 | 41 | 18 | 318 | 18.6% |
| Sum | 558 | 60 | 53 | 26 | 697 | 11.3% |

### Example 2. Developing a Database of Signatures that can be Used for Training and Developing a Machine Learning Algorithm Model with Reliable Performance in Ruling Out High Risk of Invasion or Metastasis

To develop predicative signatures for invasion/metastases, over 400 literature-derived expression-based signatures were evaluated. These different expression-based signatures cover a broad spectrum of molecular processes, including cancer hallmarks such as endothelial to mesenchymal transition, tumor microenvironment, immune-oncology, metabolism, and treatment sensitivity. Additionally, genes that were differentially expressed, variants determined via laboratory test services (e.g., Afirma) were also analyzed. Additionally, clinical variables such as nodule location, gender, cytology group were also assessed. FIG. 1 shows a general schematic of the features. Table 2 shows signatures used to generate the best models for ruling out risk of invasiveness in these studies. Each signature comprises a measure of the expression of 1 to 20,000 genes. In some instances, a signature comprises a measure of the expression of 1, 2, 3, 4, or 5 genes. In some instances, a signature comprises a measure of the expression of 6, 7, 8, 9, or 10 genes. In some instances, a signature comprises a measure of the expression of 10 to 100 genes. In some instances, a signature comprises a measure of the expression of more than 100 to 200 genes, 200 to 300 genes, 300 to 400 genes, 400 to 500 genes, 500 to 600 genes, 600 to 700 genes, 700 to 800 genes, 800 to 900 genes, or 900 to 1000 genes. In some instances, a signature comprises a measure of the expression of 1000 to 2000 genes. In some instances, a signature comprises a measure of the expression of more than 2000 genes. In some instances, a signature comprises a measure of the expression of more than 3000 genes. In some instances, a signature comprises a measure of the expression of more than 4000 genes. In some instances, a signature comprises a measure of the expression of more than 5000 genes. In some instances, a signature comprises a measure of the expression of 5000 to 10,000 genes. In some instances, a signature comprises a measure of the expression of 10,000 to 11,000 genes, 11,000 to 12,000 genes, 12,000 to 13,000 genes, 13,000 to 14,000 genes, 14,000 to 15,000 genes, 15,000 to 16,000 genes, 16,000 to 17,000 genes, 17,000 to 18,000 genes, 18,000 to 19,000 genes, or 19,000 to 20,000 genes. A measure in this context is a mathematical evaluation such as an average or a correlation, optionally with filtering of certain values.

TABLE 2
Gene signatures used for ruling out high risk of invasiveness

| Signature Name | Signature Type | Signature description |
|---|---|---|
| Keratin5/6 gene | Tumor | An average of gene expression for KRT5, KRT6A, |
| model | microenvironment | KRT6B, KRT6C |
| Lovastatin drug | Drug sensitivity | A signature reflecting the improved ClariomS |
| response score | | compatible version of a drug response model. The |
| | | original model was developed based on the NCI60 |
| | | drug response model, which calculate the |
| | | correlation of negative log2 of 50% grow inhibitory |
| | | concentration (GI50) and gene expression level |
| | | while filtering for highly positive or negative |
| | | correlated genes. |
| T cell signature | Tumor | A set of immune signatures curated to identify |
| | microenvironment | differences in the immune landscape in kidney |
| | | cancer subtypes. The signature is a simple average |
| | | of the genes for a given immune signature. |
| AMPK Complex | Metabolism | A set of metabolic signatures curated to identify |
| | | differences in the metabolic processes in kidney |
| | | cancer subtypes. The list of genes for the signature |
| | | is found in a Kidney TCGA study. The signature is |
| | | a simple average of the genes for a given metabolic |
| | | signature. |
| Perfisone drug | Drug sensitivity | A signature reflecting an improved ClariomS |
| response score | | compatible version of drug response model. The |
| | | original model was developed based on the NCI60 |
| | | drug response model, which calculate the |
| | | correlation of negative log2 of 50% grow inhibitory |
| | | concentration (GI50) and gene expression level |
| | | while filtering for highly positive or negative |
| | | correlated genes. |
| AR activity | AR biology | A signature reflecting the AR activity based on the |
| | | expression of downstream targets. Patients with low |
| | | activity at high risk of developing rapid met and |
| | | may fail ADT therapy |
| Cytotoxic T | Tumor | Tumor Immune Dysfunction and Exclusion |
| lymphocytes | microenvironment | signatures. It is a computational framework |
| | | developed to evaluate the potential of tumor |
| | | immune escape from the gene expression profiles of |
| | | cancer samples. |
| Immunophenoscore | Tumor | A signature containing determinants of tumor |
| | microenvironment | immunogenicity for use in a quantification termed |
| | | immunophenoscore. The immunophenoscore was a |
| | | superior predictor of response to anti-cytotoxic T |
| | | lymphocyte antigen-4 (CTLA-4) and anti- |
| | | programmed cell death protein 1 (anti-PD-1) |
| | | antibodies in two independent validation cohorts. |

### Example 3. Setting Up >300 Machine Learning Models

As there are multiple complex molecular processes are associated with invasion/metastases, over 300 different machine-learning (ML) models were trained within the repeated 5-fold cross validation scheme. FIG. 2 shows a schematic of the how the models were generated.

A starting feature set of clinical covariates, gene expression data and GRID Signatures, and Veracyte Thyroid signatures were used. The clinical covariates used were cohort, cytology group, BRAF status. The gene expression data was generated from expression profile coming from RNA sequencing platform involving >20,000 genes. 435 GRID Signatures were generated using a proprietary pipeline along with 5 Veracyte Thyroid signatures. GRID Signature comprises literature-derived signatures of different genes and variants relating to tumors, drug response or sensitivity, or other metabolic activities of cells. The GRID signatures are based on microarray platform, with normalized RNA-seq expression data, and the signature calculation code can be directly applied to samples sequenced using the RNA-seq platform and yielding GRID signature values. For normalization, whole-exome enriched RNA-seq assay targeting greater than 20,000 genes was analyzed. The counts from each gene measured through RNA-seq are normalized using variance stabilizing transformation (VST), yielding normalized expression values which are now approximately homoskedastic (having constant variance along the range of mean values). The transformation also normalizes with respect to technical factors like library size.

In order to select the features, a variety of feature selection methods were used. For the three components of features, Veracyte clinical covariates with either gene expression data or GRID Signature or all the features together were combined and run with one of the following feature selection methods: Differentially expressed genes/GRID Signatures, Hierarchical clustering with top 50%, 20%, 10% of features within each cluster, HOPACH clustering with top 50%, 20%, 10% of features within each cluster, and Boruta feature selection method. Additionally, gene sets related to epithelial mesenchymal transition (EMT), Risk of recurrence (ROR) and BRAF-like, RAS-like (BRS) were inputted as potential features. Table 3 shows an example list of BRS genes that can be used as features in the model.

TABLE 3
List of BRS gene features.

| | | | |
|---|---|---|---|
| ANKRD46 | CREB5 | PDLIM4 | TGFBR1 |
| CYB561 | CTSC | PLCD3 | DCSTAMP |
| GNA14 | CYP1B1 | PLEKHA6 | TMEM43 |
| HGD | DTX4 | PNPLA5 | |
| KATNAL2 | DUSP5 | PPL | |
| KCNAB1 | ETHE1 | PROS1 | |
| KCNIP3 | EVA1A | PTPRE | |
| LGI3 | FAM20C | PVRL4 | |
| MLEC | FCHO1 | RASGEF1B | |
| NQO1 | FN1 | RUNX1 | |
| SFTPC | FSTL3 | RUNX2 | |
| SLC4A4 | GABRB2 | SDC4 | |
| SORBS2 | GBP2 | SEL1L3 | |
| ABTB2 | ITGA3 | SFTPB | |
| AHR | ITGB8 | SLC35F2 | |
| ANKLE2 | KCNN4 | SOX4 | |
| ANXA1 | LAMB3 | SPOCK2 | |
| ARNTL | LLGL1 | STK17B | |
| ASAP2 | LY6E | SYT12 | |
| BID | MET | TACSTD2 | |
| CDC42EP1 | PDE5A | TBC1D2 | |

Different machine learning algorithms were used to perform machine learning training including: Penalized Generalized linear model (PGLM) and its hierarchical version, Random forest (rf), Support Vector Machine (SVM) and SVM-radial, and ensemble modeling of the above five models (3 ways of ensemble). By combining the different starting features with different feature selection and machine learning training algorithms, 432 different models were generated and evaluated.

### Example 4. Evaluation of Different Models

These different models were evaluated using Bethesda (B) V/VI and indeterminate thyroid nodules (ITN-BIII/IV) that are GSC-suspicious to determine an appropriate model. A first verification cohort was used to evaluate the models and included 146 patients, 27 (18.5%) males and 119 (81.5%) females with a mean age of 53 years [IQR: 39-61]. Eighty percent of samples were Bethesda III/IV and classified as GSC-suspicious and 20% Bethesda V/VI. Twenty-three (16%) were BRAFV600E positive. Four (2.7%) were annotated as high risk for invasion and 5 (3.4%) were annotated high risk for LNM from the surgical pathology reports. A second verification cohort was also used to evaluate the models and included 203 patients, 59 (29%) males and 144 (71. %) females with a mean age of 54 years [IQR: 40-65]. Seventy-five percent of samples were Bethesda III, twenty-five percent were Bethesda IV, and those were classified as GSC-suspicious.

TABLE 4
Clinicogenomic characteristics of verification cohorts

| | Verification | | Verification | |
|---|---|---|---|---|
| | cohort I | | cohort II | |
| Total (n) | 146 | | 203 | |
| Age (median IQR) | 53 | [39-61] | 54 | [40-65] |
| Sex | | | | |
| Male | 27 | (18.5%) | 59 | (29%) |
| Female | 119 | (81.5%) | 144 | (71%) |
| Cytology Bethesda | | | | |
| III | 83 | (56.8%) | 152 | (75%) |
| IV | 35 | (24%) | 51 | (25%) |
| V | 10 | (6.8%) | 0 | |
| VI | 18 | (12.3%) | 0 | |
| Invasion outcome | | | | |
| low risk: no invasion | 131 | (89.7%) | 170 | (83.7%) |
| Low risk: min invasion (<4 vessels) | 11 | (7.5%) | 29 | (14.3%) |
| High risk: Extensive vascular invasion | 3 | (2%) | 3 | (1.4%) |
| (>=4) | | | | |
| High risk: Extensive extra-thyroidal | 1 | (0.7%) | 1 | (0.5%) |
| Lymph node metastasis | | | | |
| Low risk: no nodes | 138 | (94.5%) | 199 | (98%) |
| Low risk: central neck <2 mm | 3 | (2%) | 2 | (1%) |
| or <40% LN involvement | | | | |
| High risk: central neck >2 mm | 3 | (2%) | 2 | (1%) |
| or >40% LN involvement | | | | |
| High risk: Lateral neck nodes | 2 | (1.4%) | 0 | |
| Variants | | | | |
| BRAFV600E | 23 | (15.7%) | 9 | (4.4%) |

Nested 5-fold cross validation (CV) was used for model training, parameters optimization to reduce overfitting, and to evaluate the model's performance. The best performing model based on the 5-fold CV was trained on the full training cohort, locked, and then tested in the preliminary verification set.

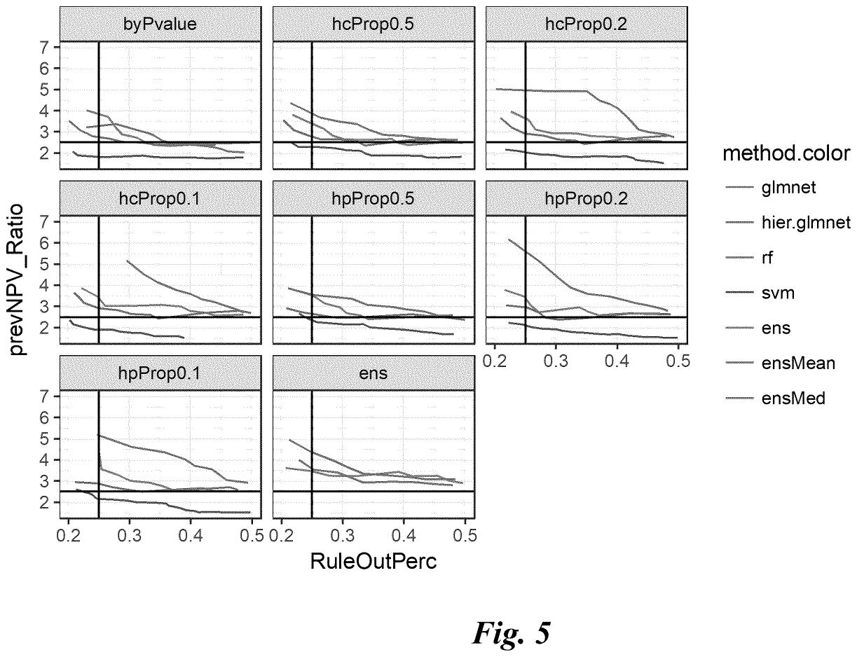

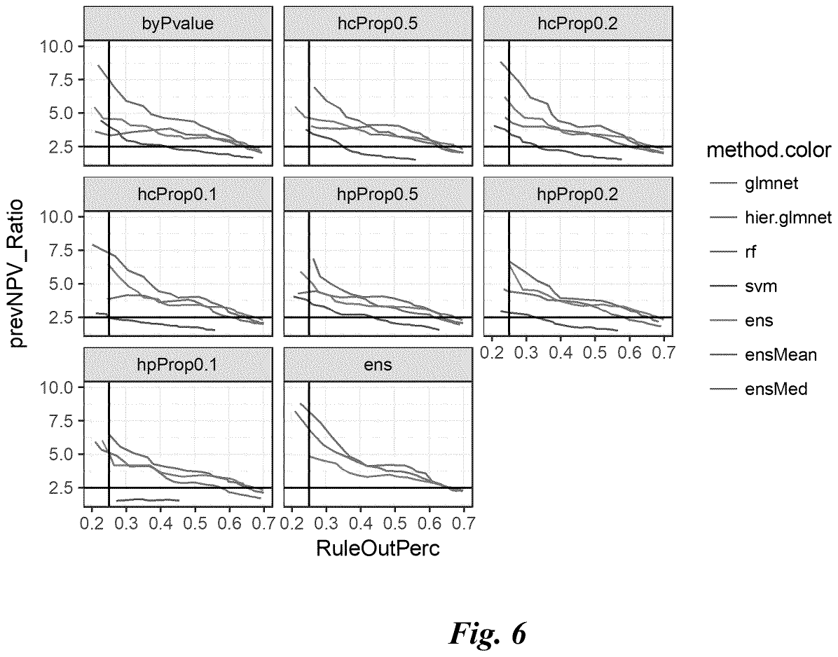

FIG. 5 shows an evaluation of different models for classification of invasiveness relative to each other based on the rule out percentage when a particular set of features and machine learning algorithms were used. Each box indicates one feature selection scheme. The PrvNPV_Ratio y-axis is calculated via prevalence divided by (1-NPV). The rule out percentage is displayed on the x-axis. The best performing model was chosen by visual inspection on each plot to find the method that dominates other methods. Additionally, the 0.3, 0.4, and 0.5 rule out percentages were evaluated and best performing methods were selected for further comparisons. Similarly. FIG. 6 shows the evaluation for models for the classification of metastases.

A composite expression signature built upon known signatures, and in the 5-fold CV results in the training cohort, a model using random forest ML algorithm was shown to be the best performing for ruling-out high-risk level 2/3 invasion. The best model included cancer pathways as features. The most important feature in this model was follicular-to-mesenchymal transition score. Other immune response related pathways and tumor microenvironment related variables were important. Cytology groups and BRAF status had very low importance in this model. The best model for invasion consists of 12 features, including 9 gene signatures and 3 covariates, including the GRID gene signatures shown in Table 2, and a signature comprising genes CDH2, TWIST1, FN1, ITGB6, DIO1, SCL5A5, and TPO, which were related to epithelial mesenchymal transition. The three covariates were cohort, cytological group, and the presence of BRAF. These features were then weighted to generate the final output.

The performance of the RF invasion model in the 5-fold CV results showed that the invasion model was able to rule out 41.3% of the population for high-risk invasion with a 97.6% negative predictive value (NPV). Higher scores predict test positivity. The model scores were significantly higher in Bethesda V/VI compared to Bethesda III/V (p<0.0001). The model scores were similar in samples from patients with no invasion and patients with minimal invasion (both labeled as low risk) (p=0.62). Within samples with Bethesda V/VI (n=312), 42 (13.7%) samples were ruled out, and within Bethesda III/IV samples (n=395), 246 (62%) were ruled out for clinically significant invasion. Rule out % was similar in males (44.7%) and females (40.5%) (Fishers exact test p=0.35) (Table 6).

In the verification cohort, the locked RF invasion model was able to rule out 43.8% with 100% NPV and specificity of 45%. The model scores were significantly higher in Bethesda V/VI compared to Bethesda III/V (p<0.0001). Within samples with Bethesda V/VI (n=28), 1 sample was ruled out, and within Bethesda III/IV samples (n=118), 64 (54%) were ruled out.

In the 5-fold CV results in the training cohort, more than 30 models were tested for metastases. The best performing model was a penalized generalized linear model that used differentially expressed genes that are related to BRAF-like variants and cytology variables. Important features in this model were BRAF-like status and the expression of ANKRD46.

Higher model scores were associated with positive test results. The model scores were significantly higher in Bethesda V/VI compared to Bethesda III/V (p<0.0001). The model scores were also significantly higher in Bethesda VI (n=200) compared to Bethesda V (n=112) (p<0.0001). The model scores were higher in samples from patients with minimal LN metastasis compared to samples with no LNM (both scored as low risk) (p<0.0001). Of samples with Bethesda V/VI (n=312), 51 (16.3%) samples were ruled out; 40% were ruled out in Bethesda V (n=112), and within Bethesda III/IV samples (n=385), 294 (76%) were ruled out (Table 6). Rule out % was similar in males (52%) and females (48%) (Fishers exact test p=0.41)

In the verification cohort, the LNM signature ruled out 51.3% with 100 NPV % and 53% specificity. The model scores were significantly higher in Bethesda V/VI compared to Bethesda III/V (p<0.0001). Within samples with Bethesda V/VI (n=28), 2 samples were ruled out (Table 6).

Table 5 lists the genes feature used in the best metastases model. Three covariates of cohort, cytological group (e.g., Bethesda category), and the presence of BRAFV600E were also additionally used, resulting in a 35 feature model. These features were then weighted to generate the final output.

TABLE 5
List of genes for metastases classifier model prediction

| | | |
|---|---|---|
| 1 | ANKRD46 | ENSG00000186106 |
| 2 | SFTPC | ENSG00000168484 |
| 3 | RUNX2 | ENSG00000124813 |
| 4 | KATNAL2 | ENSG00000167216 |
| 5 | ITGB8 | ENSG00000105855 |
| 6 | FCHO1 | ENSG00000130475 |
| 7 | EVA1A | ENSG00000115363 |
| 8 | GBP2 | ENSG00000162645 |
| 9 | DCSTAMP | ENSG00000164935 |
| 10 | SFTPB | ENSG00000168878 |
| 11 | BID | ENSG00000015475 |
| 12 | TBC1D2 | ENSG00000095383 |
| 13 | ARNTL | ENSG00000133794 |
| 14 | SPOCK2 | ENSG00000107742 |
| 15 | ANXA1 | ENSG00000135046 |
| 16 | ASAP2 | ENSG00000151693 |
| 17 | PTPRE | ENSG00000132334 |
| 18 | LAMB3 | ENSG00000196878 |
| 19 | ANKLE2 | ENSG00000176915 |
| 20 | HGD | ENSG00000113924 |
| 21 | SOX4 | ENSG00000124766 |
| 22 | PDE5A | ENSG00000138735 |
| 23 | GABRB2 | ENSG00000145864 |
| 24 | SORBS2 | ENSG00000154556 |
| 25 | CYP1B1 | ENSG00000138061 |
| 26 | PDLIM4 | ENSG00000131435 |
| 27 | LY6E | ENSG00000160932 |
| 28 | DUSP5 | ENSG00000138166 |
| 29 | KCNAB1 | ENSG00000169282 |
| 30 | PNPLA5 | ENSG00000100341 |
| 31 | LLGL1 | ENSG00000131899 |
| 32 | MET | ENSG00000105976 |

Using the models described above, 41.3% of the combined cohort was ruled out for clinically relevant invasion with a NPV of 97.6%. Similarly, 49.8% of the cohort is ruled out for clinically relevant metastases with an NPV of 98.6%.

24 samples of the training cohort were identified high risk for both invasion and LNM on surgical pathology. 19 samples were classified by both models as high risk, and only 2 were ruled out by both models.

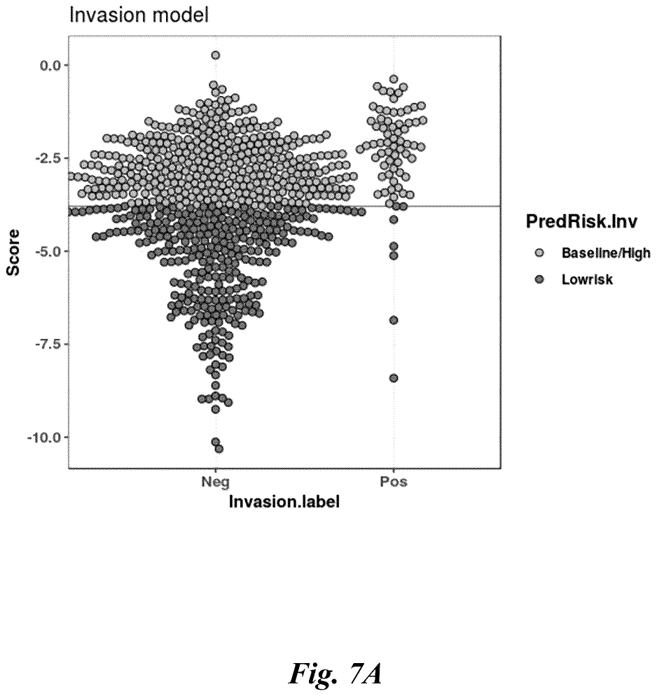

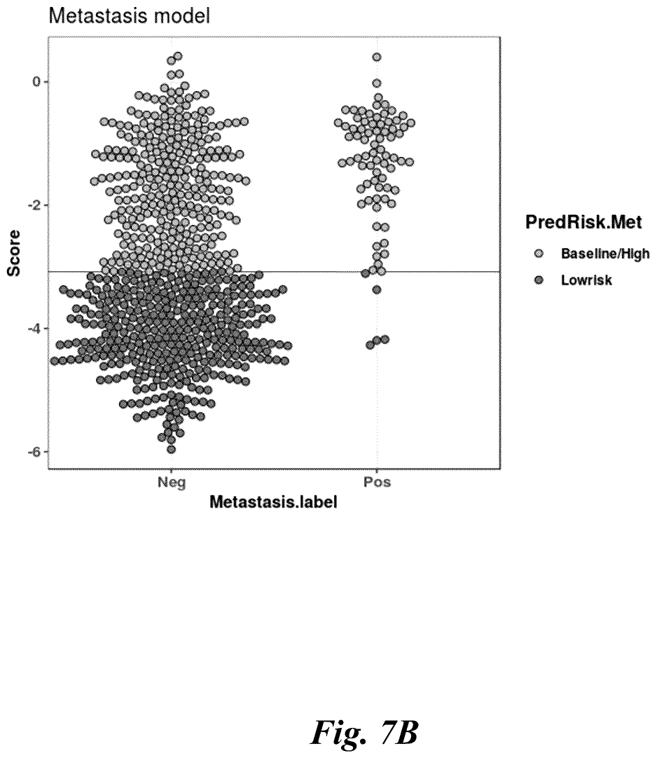

FIGS. 7A and 7B show the results of the best invasion model and best metastasis model, respectively. Each dot represents one sample. Purple dots are samples classified to be baseline or high risk, whereas green dots are classified to be low risk. The line denotes the cutoff threshold for low risk, with a sample above the line indicating a prediction of baseline or high risk, and a sample below the line indicating a prediction of low risk. This rule-out capability represents a >4-fold reduction in clinically relevant invasion and >7-fold reduction in clinically relevant metastases from the baseline prevalence (10% and 11.3% respectively). For those tumors that do not meet the rule out threshold, the risk of clinically significant invasion and metastases are at the baseline prevalence or higher.

Table 6 shows an additional breakdown of the data relating to the model. Statistics relating to specific subgroups of the provided, relating to the rule out percentage for each subgroup.

TABLE 6
Percentage (%) rule out of patients based on invasion
and LNM models across different subgroups

| | |
|---|---|
| Invasion model | LNM model |
| rule out n(%) | rule out n (%) |

Training (n = 697)

| | | | | |
|---|---|---|---|---|
| Overall (n = 697) | 288 | (41.3%) | 345 | (49.8%) |
| Bethesda III (n = 253) | 168 | (66.4%) | 194 | (76.7%) |
| Bethesda IV (n = 132) | 78 | (59.1%) | 100 | (75.7%) |
| Bethesda V (n = 112) | 20 | (17.8%) | 45 | (40%) |
| Bethesda VI (n = 200) | 22 | (11%) | 6 | (3%) |
| BRAFV600E+ | 26 | (11%) | 2 | (1%) |
| (n = 236) | | | | |
| Males (n = 152) | 68 | (44.7%) | 80 | (52.6%) |
| Females (n = 545) | 220 | (40.4%) | 264 | (48.5%) |

Verification cohort I MH (n = 146)

| Overall (n = 146) | 64 | (43.8%) | 75 | (51.3%) |
|---|---|---|---|---|
| Bethesda III (n = 83) | 49 | (59%) | 50 | (60.2%) |
| Bethesda IV (n = 35) | 14 | (40%) | 23 | (65.7%) |
| Bethesda V/VI (n = 28) | 1 | (3.5%) | 2 | (7%) |
| BRAFV600E+ | 1 | (4.3%) | 0 | |
| (n = 23) | | | | |
| Males (n = 27) | 14 | (52%) | 17 | (63%) |
| Females (n = 119) | 50 | (42%) | 58 | (49%) |

Verification cohort II BW (n = 203)

| Overall (n = 203) | 115 | (57%) | 111 | (54.7%) |
|---|---|---|---|---|
| Bethesda III (n = 152) | 91 | (60%) | 81 | (53.3%) |
| Bethesda IV (n = 51) | 24 | (47%) | 30 | (59%) |
| BRAFV600E+ | 1 | (11%) | 0 | |
| (n = 9) | | | | |
| Males (n = 59) | 32 | (54.2%) | 27 | (45.7%) |
| Females (n = 144) | 83 | (57.6%) | 84 | (58.3%) |

Models were also tested using smaller number of features. Additional metastases models were generated that used a smaller number of features than the best performing model. Models using the top 5 features (ranked by importance), top 10 features, and top 20 features were also generated and evaluated. The top 5 feature model used the 3 highest ranked genes and two clinical covariates of BRAF status (e.g., presence of BRAFV600E), and cytology group (e.g., Bethesda category). The top 5 feature model used the 8 highest ranked genes and two clinical covariates of BRAF status (e.g., presence of BRAFV600E), and cytology group (e.g., Bethesda category). The top 20 feature model used the 17 highest ranked genes and three clinical covariates of BRAF status (e.g., presence of BRAFV600E), cytology group (e.g., Bethesda category), and cohort.

Table 7 shows the performance metrics. As shown by the metrics, the original best performing model and the models using less features showed similar NPV values and sensitivity values. In some cases, such as in the top 20 features model, the NPV and sensitivity was slightly better for the model with less features. This demonstrates that different combinations and subsets of features may be used to generate functional algorithms and classifiers and that models that are accurate, sensitive, or specific (or have a desired or particular performance metric threshold) are not limited to only those deemed as the best performing models.

TABLE 7
Performance of models with less features

| | | 40th percentile | | | | 60th percentile | |
|---|---|---|---|---|---|---|---|
| | | cutoff | | Median cutoff | | cutoff | |
| | | (40% rule out) | | (50% rule out) | | (60% rule out) | |
| | | | Sensi- | | Sensi- | | Sensi- |
| Model | AUC | NPV | tivity | NPV | tivity | NPV | tivity |
| Original best | 0.812 | 98.92 | 96.2 | 98.28 | 92.41 | 97.13 | 84.8 |
| performing | | | | | | | |
| model | | | | | | | |
| Top 5 | 0.777 | 98.21 | 93.67 | 96.84 | 86.08 | 95.45 | 75.95 |
| features | | | | | | | |
| Top 10 | 0.82 | 98.92 | 96.2 | 98 | 91.14 | 96.89 | 83.54 |
| features | | | | | | | |
| Top 20 | 0.829 | 98.57 | 94.94 | 98.28 | 92.41 | 98.1 | 89.87 |
| features | | | | | | | |

### Example 5. RNA Purification

RNA was purified with the AllPrep Micro kit (Qiagen, Hilden, Germany) as previously described. RNA was quantified using the QuantiFluor RNA System (Promega, Madison, WI). Fluorescence was read with a Tecan Infinite 200 Pro plate reader (Tecan, Männedorf, Switzerland). RNA Integrity Number was determined with the Bioanalyzer 2100 (Agilent, Santa Clara, CA).

### Example 6. Library Preparation

Samples were randomized and plated into 96 well plates according to their random order. Each plate contained Universal Human Reference RNA (Agilent, Santa Clara, CA), a benign thyroid tissue control sample, a malignant thyroid tissue control sample, a medullary thyroid carcinoma tissue control sample and 6 FNAs that were run on every plate in the study. Additionally, 3 samples from each plate were randomly selected to be included as technical replicates.

15 ng of total RNA was transferred to a 96 well plate. The TruSeq RNA Access Library Preparation Kit (Illumina, San Diego, CA) was adapted for use on the Microlab STAR robotics platform (Hamilton, Reno, NV). During library preparation, total RNA is fragmented, reverse transcribed, end-repaired, A-tailed, and Illumina adapters with individual indexes are ligated. Following PCR and AMpure XP (Beckman Coulter, Indianapolis, IN) cleanup, library size and quantity was determined with the Fragment Analyzer (Advanced Analytical, Ankeny, IA). 250 ng of 4 libraries were combined and sequentially captured with the human exome to remove ribosomal RNA, intronic, and intergenic sequences. Following PCR and AMpure XP (Beckman Coulter, Indianapolis, IN) cleanup, library size and quantity were determined with the Bioanalyzer 2100 (Agilent, Santa Clara, CA).

### Example 7. Next-Generation Sequencing

Libraries were normalized to 2 nM, pooled to 16 samples per sequencing run, and denatured according to the manufacturer's instructions. 1% phiX library (Illumina, San Diego, CA) was spiked into each sequencing run. Denatured and diluted libraries were loaded onto NextSeq 500 machines (Illumina, San Diego, CA) and sequenced with a NextSeq v2 High Output 150 cycle kit (Illumina, San Diego, CA) for paired end 2×76 cycle sequencing. Sequencing runs were required to have >75% of bases ≥Q30 and <1% phiX error rate.

### Example 8. RNA Sequencing Pipeline, Feature Extraction, and Quality Control

RNA-seq data was used to generate gene expression counts, identify variants, detect fusion-pairs, and calculate loss of heterozygosity (LOH) statistics. Raw sequencing data (FASTQ file) was aligned to human reference genome assembly 37 (Genome Reference Consortium) using STAR RNA-seq aligner. Expression counts were obtained by HTSeq5 and normalized using DESeq26 accounting for sequencing depth and gene-wise variability. Variants were identified using GATK variant calling pipeline, and fusion-pairs detected using STAR-Fusion. A loss of heterozygosity (LOH) statistic at chromosome and genome level was developed using variants identified genome-wide. The statistic quantifies the magnitude of LOH by calculating the proportion of variants that have a variant allele frequency (VAF; fraction of reads carrying the alternative allele) away from 0.5 (<0.2 or >0.8) after pre-filtering of variants that has a VAF exactly at zero or one, or is located in cytoband regions exhibiting abnormal excess of LOH signatures across all training samples.

To exclude low quality samples from downstream analysis, quality metrics were evaluated against pre-specified acceptance metrics for total numbers of sequenced and uniquely mapped reads, the overall proportion of exonic reads among mapped, the mean per-base coverage, the uniformity of base coverage, and base duplication and mismatch rates. All these QC metrics were generated using RNA-SeQC. Any sample that failed a QC metric was reprocessed from total RNA through library preparation and sequencing if sufficient RNA was available. Only samples passing the quality criteria were used for downstream analysis.

Methods and systems of the present disclosure may be combined with or modified by other methods and systems, such as those described in Krane et al. (2020) “The Afirma Xpression Atlas for thyroid nodules and thyroid cancer metastases: Insights to inform clinical decision-making from a fine-needle aspiration sample” Cancer Cytopathol 128 (7): 452-9, https://www.afirma.com/the-social-butterfly/afirma-gsc-better-as-one, U.S. Pat. Nos. 8,541,170, 9,495,515, and 10,934,587, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-97. (canceled)

98. A method for processing or analyzing a tissue sample of a subject, comprising:
(a) subjecting a first portion of said tissue sample to cytological analysis that indicates that said first portion of said tissue sample comprises a thyroid tissue sample with a risk of malignancy;
(b) upon identifying said first portion of said tissue sample as comprising a thyroid tissue sample with said risk of malignancy, assaying a plurality of gene expression products from a second portion of said tissue sample to yield a first data set;
(c) in a programmed computer, using a trained algorithm to process said first data set from (b) to generate a classification of a risk of being invasive, metastatic or a combination thereof, for said second portion of said tissue sample; and
(d) outputting a report indicative of said classification of said risk of being invasive, metastatic, or a combination thereof.

99. The method of claim 98, wherein the classification of the risk of being invasive, metastatic or a combination thereof, comprises said second portion of said tissue sample as having a low risk.

100. The method of claim 98, wherein the risk of being invasive, metastatic or a combination thereof comprises said second portion of said tissue sample as not having a high risk.

101. The method of claim 98, wherein the risk of malignancy is a Bethesda III, IV, V or VI classification.

102. The method of claim 98, wherein the risk of malignancy is due to an indeterminate cytopathology.

103. The method of claim 98, wherein the risk of malignancy is determined using a genomic sequence classifier.

104. The method of claim 98, wherein having a risk of being invasive comprises having an extent of invasion that is at least one of extensive lympho-vascular invasion, focal extra-thyroidal invasion, or extensive extra-thyroidal invasion.

105. The method of claim 98, wherein having a risk of being metastatic comprises having at least one of central neck nodes with greater than 2 mm tumor deposits, greater than 40% of lymph nodes involved, or lateral neck node metastases.

106. The method of claim 98, wherein said assaying comprises assaying by sequencing, array hybridization, or nucleic acid amplification.

107. The method of claim 98, wherein said classification of said second portion of said tissue sample as having a risk of being invasive, metastatic, or a combination thereof has a negative predictive value (NPV) of at least about 90%.

108. The method of claim 98, wherein the trained algorithm is a machine learning algorithm.

109. The method of claim 108, wherein the machine learning algorithm is selected from the group consisting of a penalized generalized linear regression algorithm, hierarchical penalized linear regression algorithm, random forest algorithm, support vector machine algorithm, support vector machine-radial algorithm, and combinations thereof.

110. The method of claim 98, wherein, outputting a report further comprises outputting a severity level of invasion, metastases, or combination thereof.

111. The method of claim 98, wherein said trained algorithm processes, in said first data set, sequence information corresponding to one or more genes of Tables 3 or Table 5.

112. The method of claim 98, wherein said trained algorithm processes, in said first data set, sequence information corresponding to genes associated with a tumor microenvironment, drug sensitivity, or metabolism.

113. The method of claim 98, wherein said trained algorithm processes, in said first data set, sequence information corresponding to genes associated with androgen receptors, BRAF, or RAS.

114. The method of claim 98, wherein said trained algorithm processes, in said first data set, sequence information corresponding to one or more genes selected from the group consisting of CDH2, TWIST1, FN1, ITGB6, DIO1, SCL5A5, and TPO.

115. The method of claim 98, wherein said plurality of gene expression products comprise gene expression products corresponding to one or genes of Table 3 or Table 5.

116. The method of claim 98, wherein said plurality of gene expression products comprise gene expression products corresponding to one or more genes selected from the group consisting of CDH2, TWIST1, FN1, ITGB6, DIO1, SCL5A5, and TPO.

117. The method of claim 98, wherein said tissue sample is a fresh frozen sample or a needle aspirate sample.
